(19) United States
(12) Patent Application Publication
(10) Pub. No.: US 2026/0207703 A1
(43) Pub. Date: Jul. 23, 2026

(54) COMPOSITIONS COMPRISING OXYTOCIN AND METHODS OF TREATING OPIOID OVERDOSE

(71) Applicant: The George Washington University, Washington, DC (US)

(72) Inventors: David MENDELOWITZ, Washington, DC (US); Joan ESCOBAR, Washington, DC (US); Olga DERGACHEVA, Washington, DC (US)

(21) Appl. No.: 19/571,112

(22) Filed: Mar. 18, 2026

Related U.S. Application Data
(63) Continuation of application No. PCT/US2024/047537, filed on Sep. 19, 2024.
(60) Provisional application No. 63/540,003, filed on Sep. 22, 2023.

(51) Int. Cl. A61K 38/095; A61P 11/16

(52) U.S. Cl. CPC A61K 38/095; A61P 11/16

(57) ABSTRACT

Disclosed herein compositions comprising oxytocin and methods of using those compositions to treat and/or reverse an opioid overdose due to fentanyl+xylazine.

### I. CROSS-REFERENCE TO RELATED APPLICATION

This application is a continuation of International Application No. PCT/US2024/047537, filed on Sep. 19, 2024, which claims the benefit of priority to U.S. Provisional Application No. 63/540,003 filed Sep. 22, 2023, the disclosures of which are incorporated by reference herein in their entireties.

### II. BACKGROUND

The statistics are staggering. As of December 2023, approximately 222 people die each day from an opioid overdose. 88% of opioid-involved overdose deaths involved synthetic opioids. The primary cause of death associated with ultra-potent synthetic (UPS) opioids is opioid-induced respiratory depression (OIRD), characterized by reduced respiratory rate, decreased airflow, and central apneas, ultimately resulting in severe hypoxemia and cardio-respiratory arrest.

Naloxone, a mu-opioid receptor (MOR) antagonist, rapidly reverses OIRD. However, it has a short half-life (~1 hr.) and therefore cannot cover the entire exposure to synthetic opioids that have far longer half-lives. Naloxone does not reverse non-MOR effects of UPS opioids such as muscle rigidity, does not relieve upper airway obstruction in patients with underlying obstructive sleep apnea (OSA), and may cause pulmonary edema. In addition, naloxone blocks the analgesic effects of opioids and causes acute withdrawal that often causes agitation, vomiting, and diarrhea. Another adverse outcome following overdose resuscitation using naloxone is often described as “rage” that includes angry, hostile, combativeness, and/or aggressive outbursts with between 10.9% to 41% of all overdose reversals labelled as ‘rage’ events. Another MOR antagonist, nalmefene, has a longer half-life (8-11 h), but it also causes similar adverse effects.

Exasperating the opioid crisis is the misuse of xylazine, a veterinary tranquilizer and alpha-2 adrenergic receptor agonist that is often combined with fentanyl in illegal use. In 2022, approximately 23% of fentanyl powder and 7% of fentanyl pills seized by the DEA contained xylazine. Xylazine, when used in combination with opioids produces synergistic effects in OIRD and further exacerbates the risk of fatality, as MOR antagonists are ineffective against xylazine. Reversal agents for xylazine used in veterinary medicine (yohimbine, tolazoline) have not been proved safe or effective in humans. A nonlethal dose of xylazine (100 mg/kg) produces a synergistic interaction with fentanyl and decreased the estimated LD₅₀ dose for fentanyl by approximately 100-fold.

Desperately needed are alternative non-opioid receptor antagonist-based approaches to OIRD caused by the combination of fentanyl and xylazine.

### III. BRIEF DESCRIPTION OF THE FIGURES

FIG. 1A-FIG. 1D shows three groups of male Sprague Dawley rats (n=8) were administered intraperitoneal (IP) fentanyl (0.5 mg/kg) and xylazine (1 mg/kg) either alone or in combination. Xylazine, like fentanyl, significantly elicited respiratory depression. Oxytocin (200 nmol/kg) injected intraperitoneally 10-15 minutes after fentanyl significantly reversed OIRD and increased breathing frequency (FIG. 1A), tidal volume (FIG. 1B), and minute ventilation (FIG. 1C), compared to untreated controls. FIG. 1D shows the combination of fentanyl+xylazine was a particularly potent mix that elicits respiratory depression. While naloxone was only partially effective in reversing opioid induced respiratory depression elicited by combined fentanyl+xylazine, oxytocin potently reverses opioid induced respiratory depression elicited by combined fentanyl+xylazine (FIG. 1D).

FIG. 2A shows that oxytocin significantly diminished both the incidence of apneas and hypopneas evoked by fentanyl. FIG. 2B shows that oxytocin was significantly potent compared to naloxone in diminishing the incidence of apneas evoked by fentanyl. Respiratory function was quantified using a whole-body plethysmography system in unrestrained and freely moving animals.

FIG. 3A shows that when using a lethal dose of Fentanyl (1.3 mg/kg), the intraperitoneal injection of oxytocin (200 nmol/kg) increased the survival of experimental rats. FIG. 3B shows that the same effect is observed when fentanyl (1.3 mg/kg) was combined with xylazine (10 mg/kg).

### IV. BRIEF SUMMARY

Disclosed herein is a pharmaceutical formulation comprising oxytocin and one or more pharmaceutically acceptable carriers.

Disclosed herein is a pharmaceutical formulation comprising oxytocin, naloxone, and one or more pharmaceutically acceptable carriers.

Disclosed herein is a pharmaceutical formulation comprising oxytocin, nalmefene, and one or more pharmaceutically acceptable carriers.

Disclosed herein is a pharmaceutical formulation comprising oxytocin and one or more opioid receptor antagonists, and one or more pharmaceutically acceptable carriers.

Disclosed herein is a pharmaceutical formulation comprising oxytocin, naloxone, and one or more opioid receptor antagonists, and one or more pharmaceutically acceptable carriers.

Disclosed herein is a pharmaceutical formulation comprising oxytocin, nalmefene, and one or more opioid receptor antagonists, and one or more pharmaceutically acceptable carriers.

Disclosed herein is a pharmaceutical formulation comprising oxytocin, one or more additional therapeutic agents, and one or more pharmaceutically acceptable carriers.

Disclosed herein is a pharmaceutical formulation comprising oxytocin, naloxone, one or more additional therapeutic agents, and one or more pharmaceutically acceptable carriers.

Disclosed herein is a pharmaceutical formulation comprising oxytocin, nalmefene, one or more additional therapeutic agents, and one or more pharmaceutically acceptable carriers.

Disclosed herein is a pharmaceutical formulation comprising oxytocin, one or more opioid receptor antagonists, one or more additional therapeutic agents, and one or more pharmaceutically acceptable carriers.

Disclosed herein is a pharmaceutical formulation comprising oxytocin, naloxone, one or more additional therapeutic agents, one or more opioid receptor antagonists, and one or more pharmaceutically acceptable carriers.

Disclosed herein is a pharmaceutical formulation comprising oxytocin, nalmefene, one or more opioid receptor antagonists, one or more additional therapeutic agents, and one or more pharmaceutically acceptable carriers.

Disclosed herein is a liquid spray comprising oxytocin and one or more pharmaceutically acceptable carriers.

Disclosed herein is a liquid spray comprising oxytocin, naloxone, and one or more pharmaceutically acceptable carriers.

Disclosed herein is a liquid spray comprising oxytocin, nalmefene, and one or more pharmaceutically acceptable carriers.

Disclosed herein is a liquid spray comprising oxytocin and one or more opioid receptor antagonists, and one or more pharmaceutically acceptable carriers.

Disclosed herein is a liquid spray comprising oxytocin, naloxone, and one or more opioid receptor antagonists, and one or more pharmaceutically acceptable carriers.

Disclosed herein is a liquid spray comprising oxytocin, nalmefene, and one or more opioid receptor antagonists, and one or more pharmaceutically acceptable carriers.

Disclosed herein is a liquid spray comprising oxytocin, one or more additional therapeutic agents, and one or more pharmaceutically acceptable carriers.

Disclosed herein is a liquid spray comprising oxytocin, naloxone, one or more additional therapeutic agents, and one or more pharmaceutically acceptable carriers.

Disclosed herein is a liquid spray comprising oxytocin, nalmefene, one or more additional therapeutic agents, and one or more pharmaceutically acceptable carriers.

Disclosed herein is a liquid spray comprising oxytocin, one or more opioid receptor antagonists, one or more additional therapeutic agents, and one or more pharmaceutically acceptable carriers.

Disclosed herein is a liquid spray comprising oxytocin, naloxone, one or more additional therapeutic agents, one or more opioid receptor antagonists, and one or more pharmaceutically acceptable carriers.

Disclosed herein is a liquid spray comprising oxytocin, nalmefene, one or more opioid receptor antagonists, one or more additional therapeutic agents, and one or more pharmaceutically acceptable carriers.

Disclosed herein is a method of treating a subject, the method comprising administering to a subject experiencing an opioid overdose due to fentanyl mixed with and/or laced with and/or cut with xylazine a therapeutically effective amount of a disclosed pharmaceutical formulation.

Disclosed herein is a method of treating a subject, the method comprising administering to a subject suspected of experiencing an opioid overdose due to fentanyl mixed with and/or laced with and/or cut with xylazine a therapeutically effective amount of a disclosed pharmaceutical formulation.

Disclosed herein is a method of treating a subject, the method comprising administering to a subject experiencing opioid-induced respiratory depression (OIRD) due to fentanyl mixed with and/or laced with and/or cut with xylazine a therapeutically effective amount of a disclosed pharmaceutical formulation.

Disclosed herein is a method of treating a subject, the method comprising administering to a subject suspected of experiencing opioid-induced respiratory depression (OIRD) due to fentanyl mixed with and/or laced with and/or cut with xylazine a therapeutically effective amount of a disclosed pharmaceutical formulation.

Disclosed herein is a method of treating an opioid overdose due to fentanyl mixed with and/or laced with and/or cut with xylazine, the method comprising administering to a subject in need thereof a therapeutically effective amount of a disclosed pharmaceutical formulation.

Disclosed herein is a method of treating a suspected opioid overdose due to fentanyl mixed with and/or laced with and/or cut with xylazine, the method comprising administering to a subject in need thereof a therapeutically effective amount of a disclosed pharmaceutical formulation.

Disclosed herein is a method of reducing aggression, anger, and/or violence associated with reversal of an opioid overdose due to fentanyl mixed with and/or laced with and/or cut with xylazine, the method comprising administering to a subject in need thereof a therapeutically effective amount of a disclosed pharmaceutical formulation.

Disclosed herein is a method of treating a subject, the method comprising administering to a subject experiencing an opioid overdose due to fentanyl mixed with and/or laced with and/or cut with xylazine a therapeutically effective amount of a disclosed pharmaceutical formulation.

Disclosed herein is a method of treating a subject, the method comprising administering to a subject suspected of experiencing an opioid overdose due to fentanyl mixed with and/or laced with and/or cut with xylazine a therapeutically effective amount of a disclosed pharmaceutical formulation.

Disclosed herein is a method of treating a subject, the method comprising administering to a subject experiencing opioid-induced respiratory depression (OIRD) due to fentanyl mixed with and/or laced with and/or cut with xylazine a therapeutically effective amount of a disclosed pharmaceutical formulation.

Disclosed herein is a method of treating a subject, the method comprising administering to a subject suspected of experiencing opioid-induced respiratory depression (OIRD) due to fentanyl mixed with and/or laced with and/or cut with xylazine a therapeutically effective amount of a disclosed pharmaceutical formulation.

Disclosed herein is a method of treating an opioid overdose due to fentanyl mixed with and/or laced with and/or cut with xylazine, the method comprising administering to a subject in need thereof a therapeutically effective amount of a disclosed pharmaceutical formulation.

Disclosed herein is a method of treating a suspected opioid overdose due to fentanyl mixed with and/or laced with and/or cut with xylazine, the method comprising administering to a subject in need thereof a therapeutically effective amount of a disclosed pharmaceutical formulation.

Disclosed herein is a method of reducing aggression, anger, and/or violence associated with reversal of an opioid overdose due to fentanyl mixed with and/or laced with and/or cut with xylazine, the method comprising administering to a subject in need thereof a therapeutically effective amount of a disclosed pharmaceutical formulation.

Disclosed herein is a method of treating a subject, the method comprising administering to a subject experiencing an opioid overdose due to fentanyl mixed with and/or laced with and/or cut with xylazine a therapeutically effective amount of a disclosed pharmaceutical formulation.

Disclosed herein is a method of treating a subject, the method comprising administering to a subject suspected of experiencing an opioid overdose due to fentanyl mixed with and/or laced with and/or cut with xylazine a therapeutically effective amount of a disclosed pharmaceutical formulation.

Disclosed herein is a method of treating a subject, the method comprising administering to a subject experiencing opioid-induced respiratory depression (OIRD) due to fentanyl mixed with and/or laced with and/or cut with xylazine a therapeutically effective amount of a disclosed pharmaceutical formulation.

Disclosed herein is a method of treating a subject, the method comprising administering to a subject suspected of experiencing opioid-induced respiratory depression (OIRD) due to fentanyl mixed with and/or laced with and/or cut with xylazine a therapeutically effective amount of a disclosed pharmaceutical formulation.

Disclosed herein is a method of treating an opioid overdose due to fentanyl mixed with and/or laced with and/or cut with xylazine, the method comprising administering to a subject in need thereof a therapeutically effective amount of a disclosed pharmaceutical formulation.

Disclosed herein is a method of treating a suspected opioid overdose due to fentanyl mixed with and/or laced with and/or cut with xylazine, the method comprising administering to a subject in need thereof a therapeutically effective amount of a disclosed pharmaceutical formulation.

Disclosed herein is a method of reducing aggression, anger, and/or violence associated with reversal of an opioid overdose due to fentanyl mixed with and/or laced with and/or cut with xylazine, the method comprising administering to a subject in need thereof a therapeutically effective amount of a disclosed pharmaceutical formulation.

Disclosed herein is a method of treating a subject, the method comprising administering to a subject experiencing an opioid overdose due to fentanyl mixed with and/or laced with and/or cut with xylazine a therapeutically effective amount of a disclosed pharmaceutical formulation.

Disclosed herein is a method of treating a subject, the method comprising administering to a subject suspected of experiencing an opioid overdose due to fentanyl mixed with and/or laced with and/or cut with xylazine a therapeutically effective amount of a disclosed pharmaceutical formulation.

Disclosed herein is a method of treating a subject, the method comprising administering to a subject experiencing opioid-induced respiratory depression (OIRD) due to fentanyl mixed with and/or laced with and/or cut with xylazine a therapeutically effective amount of a disclosed pharmaceutical formulation.

Disclosed herein is a method of treating a subject, the method comprising administering to a subject suspected of experiencing opioid-induced respiratory depression (OIRD) due to fentanyl mixed with and/or laced with and/or cut with xylazine a therapeutically effective amount of a disclosed pharmaceutical formulation.

Disclosed herein is a method of treating an opioid overdose due to fentanyl mixed with and/or laced with and/or cut with xylazine, the method comprising administering to a subject in need thereof a therapeutically effective amount of a disclosed pharmaceutical formulation.

Disclosed herein is a method of treating a suspected opioid overdose due to fentanyl mixed with and/or laced with and/or cut with xylazine, the method comprising administering to a subject in need thereof a therapeutically effective amount a disclosed pharmaceutical formulation.

Disclosed herein is a method of reducing aggression, anger, and/or violence associated with reversal of an opioid overdose due to fentanyl mixed with and/or laced with and/or cut with xylazine, the method comprising administering to a subject in need thereof a therapeutically effective amount of a disclosed pharmaceutical formulation.

### V. DETAILED DESCRIPTION

The present disclosure describes formulations, compounded compositions, kits, capsules, containers, and/or methods thereof. It is to be understood that the inventive aspects of which are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those disclosed herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

### A. Definitions

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those disclosed herein can be used in the practice or testing of the present invention, example methods and materials are now described.

This disclosure describes inventive concepts with reference to specific examples. However, the intent is to cover all modifications, equivalents, and alternatives of the inventive concepts that are consistent with this disclosure.

In an aspect, the singular forms “a”, “an”, and “the” include plural referents unless the context clearly dictates otherwise.

In an aspect, the phrase ‘consisting essentially of’ limits the scope of a claim to the recited components in a composition or the recited steps in a method as well as those that do not materially affect the basic and novel characteristic or characteristics of the claimed composition or claimed method. The phrase ‘consisting of’ excludes any component, step, or element that is not recited in the claim. The phrase ‘comprising’ is synonymous with ‘including’, ‘containing’, or ‘characterized by’, and is inclusive or open-ended. ‘Comprising’ does not exclude additional, unrecited components or steps.

In an aspect, when referring to any numerical value, the term ‘about’ means a value falling within a range that is ±10% of the stated value.

Ranges can be expressed herein as from ‘about’ one particular value, and/or to ‘about’ another particular value. When such a range is expressed, a further aspect includes from the one particular value and/or to the other particular value. Similarly, when values are expressed as approximations, by use of the antecedent ‘about,’ it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as ‘about’ that particular value in addition to the value itself. For example, if the value ‘10’ is disclosed, then ‘about 10’ is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight component X and 5 parts by weight component Y, X and Y are present at a weight ratio of 2:5, and are present in such ratio regardless of whether additional components are contained in the compound.

In an aspect, the terms ‘optional’ or ‘optionally’ means that the subsequently described event or circumstance can or cannot occur, and that the description includes instances where said event or circumstance occurs and instances where it does not. In an aspect, a disclosed method can optionally comprise one or more additional steps, such as, for example, repeating an administering step or altering an administering step.

In an aspect, the term ‘subject’ refers to the target of administration, e.g., a human being. The term ‘subject’ also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, or rodent. The term does not denote a particular age or sex, and thus, adult and child subjects, as well as fetuses, whether male or female, are intended to be covered. In an aspect, a subject can be a human patient. In an aspect, a subject can have an opioid dependence, be suspected of having opioid dependence, or be at risk of developing opioid dependence. In an aspect, a subject can use illicit opioids, be suspected of using illicit opioids, or be at risk of using illicit opioids.

In an aspect, the term ‘diagnosed’ means having been subjected to an examination by a person of skill, for example, a physician, and found to have a condition that can be diagnosed or treated by one or more disclosed pharmaceutical formulations, one or more disclosed liquid sprays, one or more disclosed devices, or a combination thereof, or by one or more disclosed methods. For example, ‘diagnosed with a disease or disorder’ means having been subjected to an examination by a person of skill, for example, a physician, and found to have a condition (such as an opioid overdose due to fentanyl mixed with and/or laced with and/or cut with xylazine) that can be treated by one or more disclosed pharmaceutical formulations, one or more disclosed liquid sprays, one or more disclosed devices, or a combination thereof, or by one or more disclosed methods. For example, “suspected of having a disease or disorder” can mean having been subjected to an examination by a person of skill, for example, a physician, and found to have a condition (such as an opioid overdose due to fentanyl mixed with and/or laced with and/or cut with xylazine) that can likely be treated by one or more disclosed pharmaceutical formulations, one or more disclosed liquid sprays, one or more disclosed devices, or a combination thereof, or by one or more disclosed methods. In an aspect, an examination can be physical, can involve various tests (e.g., blood tests, genotyping, biopsies, etc.), scans (e.g., CT scans, PET scans, etc.), and assays (e.g., enzymatic assay), or a combination thereof.

In an aspect, “fentanyl+xylazine” refers to fentanyl mixed with and/or laced with and/or cut with xylazine.

A “patient” refers to a subject afflicted with a disease or disorder (e.g., an opioid overdose due to fentanyl mixed with and/or laced with and/or cut with xylazine). In an aspect, a patient can refer to a subject that has been diagnosed with or is suspected of having a disease or disorder such as opioid overdose due to fentanyl mixed with and/or laced with and/or cut with xylazine. In an aspect, a patient can refer to a subject that has been diagnosed with or is suspected of having a disease or disorder and is seeking treatment or receiving treatment for a disease or disorder (such as an opioid overdose due to fentanyl mixed with and/or laced with and/or cut with xylazine).

In an aspect, the phrase “identified to be in need of treatment for a disease or disorder,” or the like, refers to selection of a subject based upon need for treatment of the disease or disorder. For example, a subject can be identified as having a need for treatment of a disease or disorder (e.g., an opioid overdose due to fentanyl mixed with and/or laced with and/or cut with xylazine) based upon an earlier diagnosis by a person of skill and thereafter subjected to treatment for the an opioid overdose due to fentanyl mixed with and/or laced with and/or cut with xylazine. In an aspect, the identification can be performed by a person different from the person making the diagnosis. In an aspect, the administration can be performed by one who performed the diagnosis.

In an aspect, “inhibit”, “inhibiting”, and “inhibition” mean to diminish or decrease an activity, level, response, condition, severity, disease, or other biological parameter. This can include, but is not limited to, the complete ablation of the activity, level, response, condition, severity, disease, or other biological parameter. This can also include, for example, a 10% inhibition or reduction in the activity, level, response, condition, severity, disease, or other biological parameter as compared to the native or control level (e.g., a subject not having received one or more disclosed pharmaceutical formulations, one or more disclosed liquid sprays, one or more disclosed devices, or a combination thereof). Thus, in an aspect, the inhibition or reduction can be a 10%, 20%, 30%, 40%, 50%, 60%, 70%, 80%, 90%, 100%, or any amount of reduction in between as compared to native or control levels. In an aspect, the inhibition or reduction can be 10-20%, 20-30%, 30-40%, 40-50%, 50-60%, 60-70%, 70-80%, 80-90%, or 90-100% as compared to a native or control level (e.g., a subject not having received one or more disclosed pharmaceutical formulations, one or more disclosed liquid sprays, one or more disclosed devices, or a combination thereof). In an aspect, the inhibition or reduction can be 0-25%, 25-50%, 50-75%, or 75-100% as compared to native or control levels. In an aspect, a native or control level can be a pre-disease or pre-disorder level (such as a pre-opioid overdose state).

In an aspect, “treat” or “treating” or “treatment” include palliative treatment, that is, treatment designed for the relief of symptoms rather than the curing of the disease, pathological condition, or disorder (e.g., an opioid overdose due to fentanyl mixed with and/or laced with and/or cut with xylazine); preventative treatment, that is, treatment directed to minimizing or partially or completely inhibiting the development of the associated disease, pathological condition, or disorder (e.g., an opioid overdose due to fentanyl mixed with and/or laced with and/or cut with xylazine); and supportive treatment, that is, treatment employed to supplement another specific therapy directed toward the improvement of the associated disease, pathological condition, or disorder (e.g., an opioid overdose due to fentanyl mixed with and/or laced with and/or cut with xylazine). In an aspect, the terms cover any treatment of a subject, including a mammal (e.g., a human), and includes: (i) preventing the undesired physiological change, disease, pathological condition, or disorder from occurring in a subject that can be predisposed to the disease but has not yet been diagnosed as having it; (ii) inhibiting the physiological change, disease, pathological condition, or disorder, i.e., arresting its development; or (iii) relieving the physiological change, disease, pathological condition, or disorder, i.e., causing regression of the disease. For example, in an aspect, treating a disease or disorder can reduce the severity of an established a disease or disorder in a subject by 1%-100% as compared to a control (such as, for example, an individual not having an opioid overdose due to fentanyl mixed with and/or laced with and/or cut with xylazine). In an aspect, treating can refer to a 1%, 2%, 3%, 4%, 5%, 6%, 7%, 8%, 9%, 10%, 20%, 30%, 40%, 50%, 60%, 70%, 80%, 90%, or 100% reduction in the severity of a disease or disorder (e.g., an opioid overdose due to fentanyl mixed with and/or laced with and/or cut with xylazine). For example, treating a disease or disorder can reduce one or more symptoms of a disease or disorder in a subject by 1%-100% as compared to a control (such as, for example, an individual not having an opioid overdose due to fentanyl mixed with and/or laced with and/or cut with xylazine). In an aspect, treating can refer to 1%, 2%, 3%, 4%, 5%, 6%, 7%, 8%, 9%, 10%, 20%, 30%, 40%, 50%, 60%, 70%, 80%, 90%, 100% reduction of one or more symptoms of an established a disease or disorder (e.g., an opioid overdose due to fentanyl mixed with and/or laced with and/or cut with xylazine). It is understood that treatment does not necessarily refer to a cure or complete ablation or eradication of a disease or disorder (e.g., an opioid overdose due to fentanyl mixed with and/or laced with and/or cut with xylazine, or one or more symptoms of OIRD). However, in an aspect, treatment can refer to a cure or complete ablation or eradication of a disease or disorder (such as an opioid overdose due to fentanyl mixed with and/or laced with and/or cut with xylazine).

In an aspect, the term “prevent” or “preventing” or “prevention” refers to precluding, averting, obviating, forestalling, stopping, or hindering something from happening (including death), especially by advance action. It is understood that where reduce, inhibit, or prevent are used herein, unless specifically indicated otherwise, the use of the other two words is also expressly disclosed. In an aspect, preventing a disease or disorder having chromatin deregulation and/or chromatin dysregulation is intended. The words “prevent”, “preventing”, and “prevention” also refer to prophylactic or preventative measures for protecting or precluding a subject (e.g., an individual) not having a given a disease or disorder (such as an opioid overdose due to fentanyl mixed with and/or laced with and/or cut with xylazine) or related complication from progressing to that complication. In an aspect, preventing respiratory arrest, cardiac arrest, and death is intended.

In an aspect, the term “side effect” refers to adverse effects or toxic effects produced by a drug including but not limited to on a tissue or organ system. Such conditions, for example, can include sedation, dizziness, nausea, vomiting, constipation, hyperalgesia, immunologic and hormonal dysfunction, muscle rigidity, myoclonus, bowel dysfunction (e.g., anorexia, gastro-oesophageal reflux, delayed digestion, abdominal pain, bloating) respiratory depression and death.

In an aspect, the terms “opioid toxicity” or “opioid overdose” refers to the effects of opioid drugs that are toxic to a subject, resulting in effects that may be mild, moderate or severe including but not limited to respiratory depression or ventilatory depression, hypoxia, loss of consciousness, decreased respiratory rate, decreased respiratory depth, apnea, delirium, hypotension, bradycardia, decreased body temperature, urinary retention, pupil miosis, sedation, dizziness, nausea, vomiting, constipation, hyperalgesia, immunologic and hormonal dysfunction, muscle rigidity, myoclonus, and bowel dysfunction. Opioid toxicity can be assessed by any appropriate method known to a clinical physician or skilled artisan. For example, opioid toxicity can be assessed by performing a central nervous system review by assessing for confusion, altered mental state, excessive drowsiness, lethargy, stupor, slurred speech (new onset), hypoventilation, shortness of breath, apnea, hypoxia, and/or hypercapnia; and/or cardiac review by assessing for bradycardia, hypotension, and/or shock.

The term “overdose” refers to a subject that takes a dose larger than originally intended that causes in one or more adverse effects.

In an aspect, the liquid naloxone formulation is a spray. In an aspect, the liquid naloxone formulation is in a simple solution form. In an aspect, the term “simple solution” refers to a solution in which the solute(s) has fully dissolved in the solvent.

In an aspect, the term “stable” includes but is not limited physical and chemical stability.

In an aspect, the terms “administering” and “administration” refer to any method of providing one or more disclosed pharmaceutical formulations, one or more disclosed liquid sprays, one or more disclosed devices, or a combination thereof, or by one or more disclosed methods to a subject. Such methods are well known to those skilled in the art and include, but are not limited to, the following: oral administration, transdermal administration, administration by inhalation, nasal administration, topical administration, in utero administration, intratumoral administration, intrahepatic administration, intravaginal administration, ophthalmic administration, intraaural administration, transnasal administration, otic administration, intracerebral administration, rectal administration, sublingual administration, buccal administration, and parenteral administration, including injectable such as intravenous administration, intra-CSF administration, intra-arterial administration, intramuscular administration, and subcutaneous administration. Administration can also include hepatic intra-arterial administration or administration through the hepatic portal vein (HPV). Administration of one or more disclosed pharmaceutical formulations, one or more disclosed liquid sprays, one or more disclosed devices, or a combination thereof can comprise administration directly into the CNS or the PNS. Administration can be continuous or intermittent. Administration can comprise a combination of one or more routes.

In an aspect, the skilled person can determine an efficacious dose, an efficacious schedule, and an efficacious route of administration for the one or more disclosed pharmaceutical formulations, one or more disclosed liquid sprays, one or more disclosed devices, or a combination thereof to treat or prevent a disease or disorder (such as an opioid overdose due to fentanyl mixed with and/or laced with and/or cut with xylazine). In an aspect, the skilled person can also alter, change, or modify an aspect of an administering step to improve efficacy of one or more disclosed pharmaceutical formulations, one or more disclosed liquid sprays, one or more disclosed devices, or a combination thereof.

By “determining the amount” is meant both an absolute quantification of a particular analyte (e.g., biomarker for an opioid overdose due to fentanyl mixed with and/or laced with and/or cut with xylazine, for example) or a determination of the relative abundance of a particular analyte (e.g., an opioid overdose due to fentanyl mixed with and/or laced with and/or cut with xylazine). The phrase includes both direct or indirect measurements of abundance or both.

In an aspect, “modifying the method” can comprise modifying or changing one or more features or aspects of one or more steps of a disclosed method. In an aspect, a method can be altered by changing the amount of the one or more disclosed pharmaceutical formulations, one or more disclosed liquid sprays, one or more disclosed devices, or a combination thereof, or by changing the frequency of administration of the one or more disclosed pharmaceutical formulations, one or more disclosed liquid sprays, one or more disclosed devices, or a combination thereof to a subject, by changing the duration of time that one or more disclosed pharmaceutical formulations, one or more disclosed liquid sprays, one or more disclosed devices, or a combination thereof is administered to a subject, or by substituting for one or more of the disclosed components and/or reagents with a similar or equivalent component and/or reagent. The same applies to all disclosed pharmaceutical formulations, disclosed liquid sprays, disclosed devices, or a combination thereof.

In an aspect, the term “pharmaceutically acceptable carrier” refers to sterile aqueous or nonaqueous solutions, dispersions, suspensions, or emulsions, as well as sterile powders for reconstitution into sterile injectable solutions or dispersions just prior to use. Examples of suitable aqueous and nonaqueous carriers, diluents, solvents, or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. In an aspect, a pharmaceutical carrier employed can be a solid, liquid, or gas. In an aspect, examples of solid carriers can include lactose, terra alba, sucrose, talc, gelatin, agar, pectin, acacia, magnesium stearate, and stearic acid. In an aspect, examples of liquid carriers can include sugar syrup, peanut oil, olive oil, and water. In an aspect, examples of gaseous carriers can include carbon dioxide and nitrogen. In preparing a disclosed composition for oral dosage form, any convenient pharmaceutical media can be employed. For example, water, glycols, oils, alcohols, flavoring agents, preservatives, coloring agents and the like can be used to form oral liquid preparations such as suspensions, elixirs, and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents, and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

In an aspect, the term “excipient” refers to an inert substance which is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See, also, for reference, Remington's Pharmaceutical Sciences, (1990) Mack Publishing Co., Easton, Pa., which is hereby incorporated by reference in its entirety.

In an aspect, “concurrently” means (1) simultaneously in time, or (2) at different times during the course of a common treatment schedule.

In an aspect, the term “contacting” refers to bringing one or more disclosed pharmaceutical formulations, one or more disclosed liquid sprays, one or more disclosed devices, or a combination thereof together with a target area or intended target area in such a manner that the one or more disclosed pharmaceutical formulations, one or more disclosed liquid sprays, one or more disclosed devices, or a combination thereof can exert an effect on the intended target or targeted area either directly or indirectly. A target area or intended target area can be one or more of a subject's organs (e.g., lungs, heart, liver, kidney, brain, etc.) affected by an opioid overdose due to fentanyl mixed with and/or laced with and/or cut with xylazine. In an aspect, a target area or intended target area can be any cell or any organ infected by a disease or disorder (such as opioid overdose due to fentanyl mixed with and/or laced with and/or cut with xylazine). In an aspect, a target area or intended target area can be any organ, tissue, or cells that are affected by a disease or disorder (such as an opioid overdose due to fentanyl mixed with and/or laced with and/or cut with xylazine).

In an aspect, a unit used to measure the activity of many vitamins, hormones, enzymes, and drugs. In an aspect, an “International Unit” is the amount of a substance that has a certain biological effect.

In an aspect, “determining” can refer to measuring or ascertaining the presence and severity of a disease or disorder, such as, for example, an opioid overdose due to fentanyl mixed with and/or laced with and/or cut with xylazine. Methods and techniques used to determine the presence and/or severity of a disease or disorder are typically known to the medical arts. For example, the art is familiar with the ways to identify and/or diagnose the presence, severity, or both of a disease or disorder (such as, for example, an opioid overdose due to fentanyl mixed with and/or laced with and/or cut with xylazine).

In an aspect, “effective amount” and “amount effective” can refer to an amount that is sufficient to achieve the desired result such as, for example, the treatment and/or prevention of a disease or disorder (e.g., an opioid overdose due to fentanyl mixed with and/or laced with and/or cut with xylazine) or a suspected disease or disorder (e.g., an opioid overdose due to fentanyl mixed with and/or laced with and/or cut with xylazine), the terms “effective amount” and “amount effective” can refer to an amount that is sufficient to achieve the desired an effect on an undesired condition (e.g., an opioid overdose due to fentanyl mixed with and/or laced with and/or cut with xylazine). For example, a “therapeutically effective amount” refers to an amount that is sufficient to achieve the desired therapeutic result or to have an effect on undesired symptoms, but is generally insufficient to cause adverse side effects. In an aspect, “therapeutically effective amount” means an amount of one or more disclosed pharmaceutical formulations, one or more disclosed liquid sprays, one or more disclosed devices, or a combination thereof that (i) treats the particular disease, condition, or disorder (e.g., an opioid overdose due to fentanyl mixed with and/or laced with and/or cut with xylazine), (ii) attenuates, ameliorates, or eliminates one or more symptoms of the particular disease, condition, or disorder e.g., an opioid overdose due to fentanyl mixed with and/or laced with and/or cut with xylazine), or (iii) delays the onset of one or more symptoms of a disclosed disease, condition, or disorder (e.g., an opioid overdose due to fentanyl mixed with and/or laced with and/or cut with xylazine). The specific therapeutically effective dose level for any particular patient will depend upon a variety of factors including the disorder being treated and the severity of the disorder; one or more disclosed pharmaceutical formulations, one or more disclosed liquid sprays, one or more disclosed devices, or a combination thereof employed; the disclosed methods employed; the age, body weight, general health, sex and diet of the patient; the time of administration; the route of administration; the rate of excretion of the one or more disclosed pharmaceutical formulations, one or more disclosed liquid sprays, one or more disclosed devices, or a combination thereof employed; the duration of the treatment; drugs used in combination or coincidental with the one or more disclosed pharmaceutical formulations, one or more disclosed liquid sprays, one or more disclosed devices, or a combination thereof employed, and other like factors well known in the medical arts. For example, it is well within the skill of the art to start doses of the one or more disclosed pharmaceutical formulations, one or more disclosed liquid sprays, one or more disclosed devices, or a combination thereof at levels lower than those required to achieve the desired therapeutic effect and to gradually increase the dosage until the desired effect is achieved. If desired, then the effective daily dose can be divided into multiple doses for purposes of administration. Consequently, a single dose of a disclosed pharmaceutical formulations, a disclosed liquid spray, a disclosed device, or a combination thereof can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition, such as, for example, an opioid overdose due to fentanyl mixed with and/or laced with and/or cut with xylazine.

In an aspect, the term “pre-primed” refers to a device, such as a nasal spray which can deliver a pharmaceutical composition to a patient in need thereof with the first actuation of the spray pump, i.e., without the need to prime the pump prior to dosing, such as by actuating the pump one or more times until a spray appears.

In an aspect, the term “actuation” refers to operation of the device such that the pharmaceutical composition is delivered therefrom.

In an aspect, the term “prone” refers to a patient who is lying face down.

In an aspect, the term “protective packaging” refers to overwrap.

The term “receptor binding or occupancy” refers to a characterization of the kinetics between a radioactive drug and receptors or other binding sites throughout the body, and characterization of the radioactive drug binding affinity to these receptors.

In an aspect, the term “recovery position” means a position of the human body in which a patient lies on his/her side, with a leg or knee out in front (e.g., to prevent rolling onto his/her stomach) and at least one hand supporting the head (e.g., to elevate the face to facilitate breathing and prevent inhalation of vomit).

In an aspect, the phrase “opioid experienced” refers to a subject that has taken an opioid at least once prior to the instance for which treatment is sought. In an aspect, a subject can be opioid experienced and non-dependent suffering from opioid toxicity or opioid overdose. In an aspect, a subject can be opioid experienced and opioid dependent suffering from opioid toxicity or opioid overdose. In an aspect, opioid experienced and non-dependent subjects can be those subjects who have used opioid drugs in the past and have not developed tolerance or dependence to the opioid drugs and can be treated for opioid toxicity or overdose. In an aspect, opioid experienced and opioid-dependent subjects can be those subjects who have used opioid drugs in the past and might have developed tolerance or dependence to the opioid drugs and can be treated for opioid toxicity or overdose.

In an aspect, “opioid management” can refer to the use of prescribed opioid medications under the medical guidance of physicians and healthcare professionals. In an aspect, the term “detoxification” refers to the process of ridding the body of a drug or its metabolites, which can be done through medically-assisted management of withdrawal symptoms.

In an aspect, the term “package insert” is used to refer to instructions customarily included in commercial packages of therapeutic products, that contain information about the indications, usage, dosage, administration, contraindications and/or warnings concerning the use of such therapeutic products.

In an aspect, the term “in combination” in the context of the administration of other therapies (e.g., other agents) includes the use of more than one therapy (e.g., drug therapy). Administration “in combination with” one or more further therapeutic agents include simultaneous (e.g., concurrent) and consecutive administration in any order. The use of the term “in combination” does not restrict the order in which therapies are administered to a subject. By way of non-limiting example, a first therapy (e.g., one or more disclosed pharmaceutical formulations, one or more disclosed liquid sprays, one or more disclosed devices, or a combination thereof) can be administered prior to (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 8 weeks, 8 weeks, 9 weeks, 10 weeks, 11 weeks, or 12 weeks), concurrently, or after (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 7 weeks, 8 weeks, 9 weeks, 10 weeks, 11 weeks, or 12 weeks or longer) the administration of a second therapy to a subject having or diagnosed an opioid overdose due to fentanyl mixed with and/or laced with and/or cut with xylazine.

In an aspect, when ranges of values are disclosed, and the notation “from n₁ . . . to n₂” or “between n₁ . . . and n₂” is used, where n1 and n2 are the numbers, then unless otherwise specified, this notation is intended to include the numbers themselves and the range between them. In an aspect, this range may be integral or continuous between and including the end values. By way of example, in an aspect, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

In an aspect, the term “about,”, is intended to qualify the numerical values which it modifies, denoting such a value as variable within a range. When no range, such as a margin of error or a standard deviation to a mean value given in a chart or table of data, is recited, the term “about” should be understood to mean the greater of the range which would encompass the recited value and the range which would be included by rounding up or down to that figure as well, considering significant figures, and the range which would encompass the recited value plus or minus 20%.

In an aspect, “absorption enhancer,” refers to a functional excipient included in formulations to improve the absorption of a pharmacologically active drug. This term usually refers to an agent whose function is to increase absorption by enhancing nasal mucous-membrane permeation, rather than increasing solubility. As such, such agents are sometimes called permeation enhancers. In particular, disclosed absorption enhancers herein may improve paracellular transport (i.e., passage through intercellular spaces and tight junctions), transcellular transport (i.e., passive diffusion or active transport across cellular membranes), or transcytosis (i.e., cellular vesicular uptake).

In an aspect, “active ingredient” or “pharmaceutically active compound” is defined in the context of a “pharmaceutical formulation” and is intended to mean a component of a pharmaceutical formulation that provides the primary pharmacological effect, as opposed to an “inactive ingredient”, which would generally be recognized as providing no pharmaceutical benefit.

In an aspect, “actuation” refers to operation of the device such that the pharmaceutical formulation is delivered therefrom.

In an aspect, “agonist” refers to a moiety that interacts with, and activates, a receptor and thereby initiates a physiological or pharmacological response characteristic of that receptor. In an aspect, “antagonist” refers to a moiety that competitively binds to a receptor at the same site as an agonist (for example, the endogenous ligand), but which does not activate the intracellular response initiated by the active form of the receptor and can thereby inhibit the intracellular responses by an agonist or partial agonist. In an aspect, an antagonist does not diminish the baseline intracellular response in the absence of an agonist or partial agonist. In an aspect, “inverse agonist” refers to a moiety that binds to the endogenous form of the receptor or to the constitutively activated form of the receptor and which inhibits the baseline intracellular response initiated by the active form of the receptor below the normal base level of activity which is observed in the absence of an agonist or partial agonist.

In an aspect, “antimicrobial preservative” refers to a pharmaceutically acceptable excipient with antimicrobial properties which is added to a pharmaceutical composition to maintain microbiological stability.

In an aspect, “AUC” refers to the area under the drug plasma concentration-time curve. The term “AUC₀₋ₜ,”, refers to the area under the drug plasma concentration-time curve from t=0 to the last measurable concentration. The term “AUC_{0-∞},”, refers to the area under the drug plasma concentration-time curve extrapolated to co. The term “AUC_{0-t/D},”, refers to the AUC₀₋ₜ normalized to 0.4 mg IN nalmefene. The term “AUC_{0-∞/D},”, refers to the AUC_{0-∞} normalized to 1.5 mg IM nalmefene.

In an aspect, “bioavailability (F)” refers to the fraction of a dose of drug that is absorbed from its site of administration and reaches, in an unchanged form, the systemic circulation. In an aspect, “absolute bioavailability” is used when the fraction of absorbed drug is related to its IV bioavailability. It can be calculated using the following formula:

$$F = ( {AUC}_{extravascular} / {AUC}_{intravenous} ) \times ( {Dose}_{intravenous} / {Dose}_{extravascular} )$$

In an aspect, “relative bioavailability (Frei)” is used to compare two different extravascular routes of drug administration and it may be calculated using the following formula:

$$Frei = ( {AUC}_{extravascular 1} / {AUC}_{extravascular 2} ) \times ( {Dose}_{extravascular 2} / {Dose}_{extravascular 1} )$$

In an aspect, “clearance (CL)” refers to the rate at which a drug is eliminated divided by its plasma concentration, giving a volume of plasma from which drug is completely removed per unit of time. CL is equal to the elimination rate constant (k) multiplied by the volume of distribution (V_{d}), wherein “V_{d}” is the fluid volume that would be required to contain the amount of drug present in the body at the same concentration as in the plasma. The term “apparent clearance (CL/F)” refers to clearance that does not take into account the bioavailability of the drug. It is the ratio of the dose over the AUC.

In an aspect, “Cₘₐₓ” refers to the maximum observed plasma concentration.

In an aspect, “coefficient of variation (CV)” refers to the ratio of the sample standard deviation to the sample mean. It is often expressed as a percentage.

In an aspect, “confidence interval” refers to a range of values which will include the true average value of a parameter a specified percentage of the time.

In an aspect, the term “device” refers to an apparatus capable of delivering a drug to subject in need thereof.

In an aspect, “delivery time” refers to the amount of time that elapses between a determination made by a healthcare professional, first responder, law enforcement (e.g., police, customs, and border patrol agents), military personnel, or an untrained individual, that an individual is in need of nasal delivery of an opioid antagonist and completion of the delivery.

In an aspect, “disease” is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

In an aspect, the term “drug raid” refers to the entry into and investigation of a facility or other site in which opioids are manufactured, stored, and/or distributed. It typically connotes such a facility/site which is, or is believed to be, in violation of the laws regulating controlled substances.

In an aspect, “elimination rate constant (λ)” refers to the fractional rate of drug removal from the body. This rate is constant in first-order kinetics and is independent of drug concentration in the body. λ is the slope of the plasma concentration-time line (on a logarithmic y scale). In an aspect, the term “λ_{z}” refers to the terminal phase elimination rate constant, wherein the “terminal phase” of the drug plasma concentration-time curve is a straight line when plotted on a semi-logarithmic graph. In an aspect, the terminal phase is often called the “elimination phase” because the primary mechanism for decreasing drug concentration during the terminal phase is drug elimination from the body. The distinguishing characteristic of the terminal elimination phase is that the relative proportion of drug in the plasma and peripheral volumes of distribution remains constant. During this “terminal phase” drug returns from the rapid and slow distribution volumes to the plasma, and is permanently removed from the plasma by metabolism or renal excretion.

In an aspect, “excipient” refers to a natural or synthetic substance formulated alongside the active ingredient of a medication, included for long-term stabilization, bulking up solid formulations, or to confer a therapeutic enhancement on the active ingredient in the final dosage form, such as facilitating drug absorption, reducing viscosity, or enhancing solubility.

In an aspect, “filled” refers to an association between a device and a pharmaceutical composition, for example, when a disclosed pharmaceutical formulation comprising a therapeutically effective amount of an opioid antagonist is present within a reservoir that forms a part of a disclosed device.

In an aspect, the term “hydrate” refers to a disclosed opioid antagonist or a salt thereof that further includes a stoichiometric or non-stoichiometric amount of water bound by non-covalent intermolecular forces.

In an aspect, “in need of treatment” and “in need thereof” when referring to treatment are used interchangeably and refer to a judgment made by a caregiver (e.g., physician, nurse, nurse practitioner, medic, or other professional assessing a potential medical hazard and planning for), that a subject will benefit from treatment. An individual “who is at risk for incidental opioid overdose”, includes an individual who accidentally ingests opioids or who intentionally ingests opioids.

In an aspect, “nalmefene” refers to 17-cyclopropylmethyl-4,5α-epoxy-6-methylenemorphinan-3,14-diol. Nalmefene is commercially available as a hydrochloride salt and is a 6-methylene analog of naltrexone. Nalmefene hydrochloride is approved for opioid overdose reversal, and as disclosed herein, can be used to prevent incidental exposure to an opioid agonist and can be administered quickly and easily as a prophylactic measure by first responders, law enforcement (e.g., police, customs, and border patrol agents) and military personnel if contact with opioids, especially high potency synthetics, is either anticipated or suspected. In an aspect, Nalmefene hydrochloride (CAS Reg. No. 58895-64-0) has been marketed under the trade names Nalmetrene®, Cervene®, Revex®, Arthrene®, and Incystene®.

In an aspect, “nostril” is synonymous with “naris”.

In an aspect, “opioid overdose” refers to an acute medical condition caused by incidental exposure to an opioid agonist in a subject. Symptoms of opioid overdose include including respiratory depression (including postoperative opioid respiratory depression, acute lung injury, and aspiration pneumonia), central nervous system depression (which may include sedation, altered level consciousness, miotic (constricted) pupils), and cardiovascular depression (which may include hypoxemia and hypotension). Visible signs of opioid overdose or suspected opioid overdose include: unresponsiveness and/or loss of consciousness (will not or cannot respond to stimuli such as shouting, shaking, or rubbing knuckles on sternum); slow, erratic, or stopped breathing; slow, erratic, or stopped pulse; deep snoring or choking/gurgling sounds; blue or purple fingernails or lips; pale and/or clammy face; slack or limp muscle tone; contracted pupils; and vomiting.

In an aspect, the term “pre-primed” refers to a device, such as a nasal spray which can deliver a pharmaceutical formulation to a subject in need thereof with the first actuation of the spray pump, i.e., without the need to prime the pump prior to dosing, such as by actuating the pump one or more times until a spray appears.

In an aspect, “protective packaging” refers to overwrap.

In an aspect, “receptor binding or occupancy” refers to a characterization of the kinetics between a radioactive drug and receptors or other binding sites throughout the body, and characterization of the radioactive drug binding affinity to these receptors.

In an aspect, “providing” in the context of providing a co-packaged drug product as disclosed herein to an individual includes co-packaging the drug product, prescribing the co-packaged drug product, and dispensing the co-packaged drug product. The providing may be done either directly to an individual (for example, to an individual for whom an opioid agonist prescription is appropriate, or who is otherwise at risk of opioid overdose) or to a second individual.

In an aspect, “solvate” refers to a disclosed opioid antagonist or a salt, thereof, that further includes a stoichiometric or non-stoichiometric amount of a solvent bound by non-covalent intermolecular forces. Preferred solvents are volatile, non-toxic, and/or acceptable for administration to humans in trace amounts.

In an aspect, the term “sterile filling,” refers methods of manufacturing of a disclosed device and a disclosed pharmaceutical formulation, such that the use of preservatives is not required. Sterile drug products may be produced using aseptic processing or terminal sterilization. Terminal sterilization usually involves filling and sealing product containers under high-quality environmental conditions. In an aseptic process, the drug product, container, and closure are first subjected to sterilization methods separately, as appropriate, and then brought together.

In an aspect, the term “storage-stable,” refers to a pharmaceutical formulation in which at least about 90% to 99.5% of the active ingredient remains in an undegraded state after storage of the pharmaceutical formulation at specified temperature and humidity for a specified time, for example, for 12 months at 25° C. and 60% relative humidity.

In an aspect, the term “substantially free of antimicrobial preservatives” is understood by one of ordinary skill in the art to describe a pharmaceutical formulation that comprises less than 1% w/w antimicrobial preservatives.

In an aspect, the term “supine” refers to a subject who is lying face up.

In an aspect, “prophylaxis” is synonymous with “prevention,” and is to be considered in its broadest context and refers to any medical or public health procedure employed to prevent a disease or condition, such as opioid overdose or a symptom thereof from occurring. It does not necessarily mean that the subject will not eventually contract a disease or condition. In an aspect, prophylaxis may include the mitigation or amelioration of the symptoms of a disease or condition or preventing or reducing the risk of developing a disease or condition or symptoms thereof. In an aspect, “prophylaxis” may include reducing the severity of the onset of a disease or condition.

In an aspect, “therapeutically effective amount” or “therapeutically effective dose” means an amount effective of one or more disclosed pharmaceutical formulations, one or more disclosed liquid sprays, one or more disclosed devices, or a combination thereof to prevent opioid overdose or symptoms thereof caused by incidental exposure of an opioid agonist in a subject. Such an amount or dose may therefore also be referred to as a “prophylactically effective” amount or dose. Opioid overdose may be moderate, severe, or even fatal. In an aspect, a treatment or pharmaceutical formulation will be therapeutically effective to prevent even moderate opioid overdose, wherein a subject is temporarily physically or mentally impaired by an opioid agonist but is in no danger of impairment or harm beyond the agonist's excretion from the subject. In an aspect, a treatment or formulation will be therapeutically effective to prevent severe overdose, wherein a subject is in danger of lasting or even permanent harm. In an aspect, one or more disclosed pharmaceutical formulations, one or more disclosed liquid sprays, one or more disclosed devices, or a combination thereof will be therapeutically effective to prevent fatal overdose.

In an aspect, “exposure” refers to an actual or anticipated contact between the subject and an opioid agonist. In an aspect, actual exposure refers to exposure that in fact occurs whether known or unknown. Anticipated exposure refers to any level of expected possibility of being exposed to an opioid.

In an aspect, “incidental exposure to opioid agonist by a subject” means that the exposure to opioid agonist is not voluntary and/or intended to self-intoxicate, but occurs as part of the subject's activities in proximity to, or potential proximity to, the opioid agonist. In an aspect, workers acting in capacity as law enforcement, inspectors, public health field agents, may face the risk of incidental exposure during activities such as cleaning or performing inspections or investigations for and/or handling opioid agonists or materials that are associated with opioid agonists, such as laboratory equipment, containers, needles, pipes, or other objects. Additionally, military, law enforcement, or security personnel may face incidental exposure to opioid agonist when a third party deliberately delivers an opioid agonist to incapacitate said personnel, for example as a gas. In an aspect, incidental exposure to opioid agonist includes, for example, exposure by inhalation to aerosolized opioid agonist and incidental transdermal exposure to opioid agonist.

In an aspect, the term “t_{1/2}” or “half-life,” refers to the amount of time required for half of a drug (for example, an opioid or an opioid antagonist) to be eliminated from the body or the time required for a drug concentration to decline by half.

In an aspect, the term “tonicity agent,” refers to a compound which modifies the osmolality of a formulation, for example, to render it isotonic. Tonicity agents include, dextrose, lactose, sodium chloride, calcium chloride, magnesium chloride, sorbitol, sucrose, mannitol, trehalose, raffinose, polyethylene glycol, hydroxyethyl starch, glycine and the like.

In an aspect, the term “tomography,”, refers to a process of imaging by sections. The images may be looked at individually, as a series of two-dimensional slices or together, as a computer-generated three-dimensional representation.

In an aspect, the term “Tₘₐₓ,” refers to the time from administration of a disclosed pharmaceutical formulations to maximum drug plasma concentration.

In an aspect, the term “untrained individual” refers to an individual administering to subject an opioid antagonist using a disclosed device, wherein the individual is not a healthcare professional and has received no training in the use of the device.

In an aspect, Cₘₐₓ refers to maximum plasma concentration, observed by inspection of individual study participant plots of plasma concentration versus time. In an aspect, Cₘₐₓ/Dose refers to Cₘₐₓ adjusted for the nominal administered dose. In an aspect, Tₘₐₓ refers to time of maximum observed concentration, obtained directly from the observed concentration versus time data.

In an aspect, AUC₀₋ₜ refers to the area under the concentration-time curve from time 0 (pre-dose) to the time of last quantifiable concentration, calculated by the linear-up/log-down trapezoidal method. In an aspect, AUC₀₋ₜ/Dose refers to AUC₀₋ₜ adjusted for the nominal administered dose. In an aspect, AU_{0-inf} refers to the area under the concentration-time curve from time 0 extrapolated to infinity, calculated as AUC₀₋ₜ+Clast/λz, where Clast is the observed last quantifiable concentration. In an aspect, AUC_{0-inf}/Dose AUC_{0-inf} adjusted for the nominal administered dose. In an aspect, AUC %_{Extrap} refers to the percentage of AUC_{0-inf} obtained by extrapolation, calculated as [(AUC_{0-inf}−AUC₀₋ₜ)/AUC_{0-inf}]*100.

In an aspect, ∧z refers to λz is the terminal-phase elimination rate constant, estimated by linear regression of logarithmically-transformed concentration versus time data. In an aspect, t_{1/2} refers to the terminal phase half-life for drug concentrations in plasma is calculated as: t_{1/2}=ln (2)/λz.

In an aspect, CL/F refers to apparent total body clearance is calculated as CL/F=Dose/AUC0-inf. In an aspect, Relative Ratio of dose-adjusted AUCinf following IN administration relative Bioavailability to dose-adjusted AUCinf following IM administration.

In an aspect, tidal volume (VT) is defined as the volume of gas entering and leaving the respiratory tract with each breath. This is a dynamic measurement and varies with the activity of the baby. For infants, tidal volume is usually expressed in units of mL. In an aspect, minute volume is the total volume of gas expired over a period of 1 minute. Accurate measurement of VT (no leak around endotracheal tube or face mask) and respiratory frequency (fR) allows estimation of minute ventilation as the product: VT fR; and is usually expressed in units of L/minute, or normalized for body weight as (L/minute)/kg.

In an aspect, functional residual capacity (FRC) is the volume of air in the lungs at the end of expiration of a normal, resting, tidal volume. Lung volume at FRC is maintained by the opposing forces of lung elastic recoil and chest wall outward recoil.

In an aspect, residual volume is the volume of air remaining in the respiratory system at the end of a maximum possible expiration.

In an aspect, thoracic gas volume (TGV) is the total amount of gas in the lung at end expiration, irrespective of whether the gas is in communication with the airways. In healthy infants without air trapping, FRC is equivalent to TGV.

In an aspect, expiratory reserve volume is the volume of air that is the difference between FRC and residual volume.

In an aspect, total lung capacity is the volume of air that is in the respiratory system at the end of a maximum possible inspiration.

Disclosed are the components to be used to prepare the disclosed isolated nucleic acid molecules, disclosed vectors, or disclosed pharmaceutical formulations as well as the disclosed isolated nucleic acid molecules, disclosed vectors, or disclosed pharmaceutical formulations used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and disclosed. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific claimed compositions and/or claimed methods.

### B. Opioids

Opioid receptors are G protein-coupled receptors (GPCRs) that are activated both by endogenous opioid peptides and by clinically important alkaloid analgesic drugs such as morphine. Opioids depress respiration, which is controlled principally through medullary respiratory centers with peripheral input from chemoreceptors and other sources. Opioids produce inhibition at the chemoreceptors via μ-opioid receptors and in the medulla via μ- and δ-opioid receptors. While there are a number of neurotransmitters mediating the control of respiration, glutamate and γ-aminobutyric acid (GABA) are the major excitatory and inhibitory neurotransmitters, respectively. This explains the potential for interaction of opioids with benzodiazepines and alcohol: both benzodiazepines and alcohol facilitate the inhibitory effect of GABA at the GABA_{A} receptor, while alcohol also decreases the excitatory effect of glutamate at NMDA receptors. Oxycodone and other opioid painkillers, as well as heroin and methadone are all implicated in fatal overdose. Heroin has three metabolites with opioid activity. Variation in the formation of these metabolites due to genetic factors and the use of other drugs could explain differential sensitivity to overdose. Metabolites of methadone contribute little to its action. However, variation in rate of metabolism due to genetic factors and other drugs used can modify methadone concentration and hence overdose risk. The degree of tolerance also determines risk. Tolerance to respiratory depression is less than complete, and may be slower than tolerance to euphoric and other effects. One consequence of this may be a relatively high risk of overdose among experienced opioid users. While agonist administration modifies receptor function, changes (usually in the opposite direction) also result from use of antagonists, for example, super sensitivity to opioids following a period of administration of antagonists such as naltrexone.

There are three principal types of opioid receptors: the δ-opioid receptor, the κ-opioid receptor, and the μ-opioid receptor. Opioid receptors are G protein-coupled receptors (GPCRs) that are activated both by endogenous opioid peptides, by clinically important alkaloid analgesic drugs such as morphine, and by synthetic analgesics such as methadone and fentanyl.

In an aspect, opioids can comprise alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, etorphine, dihydroetorphine, fentanyl and derivatives, heroin, hydrocodone, hydromorphone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphine, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propoxyphene, sufentanil, tilidine, tramadol, homologues thereof, analogs thereof, and any mixtures thereof, or any combination thereof.

In the United States, mortality rates closely correlate with opioid sales. In 2008, approximately 36,450 people died from drug overdoses. At least 14,800 of these deaths involved prescription opioid analgesics. Moreover, according to the Substance Abuse and Mental Health Services Administration, the number/rate of Americans 12 years of age and older who currently abuse pain relievers has increased by 20 percent between 2002 and 2009. In New York City, between 1990 and 2006, the fatality rate from prescription opioids increased seven-fold, from 0.39 per 100,000 persons to 2.7. Drugs classed as prescription opioids in this study include both typical analgesics, such as OxyContin® (oxycodone HCl controlled-release) and methadone (used in the treatment of dependence on other opioids such as heroin and also prescribed for pain), but the increase in the rate of drug overdose over the 16 years of the study was driven entirely by overdoses of typical analgesics. Over the same period, methadone overdoses remained stable, and overdoses from heroin declined. Whites were more likely than blacks and Latinos to overdose on these analgesics, and deaths mostly occurred in neighborhoods with lower rates of poverty, suggesting differential access to doctors who can write painkiller prescriptions may be a driving force behind the racial disparity (Cerdá et al. (2013) Drug and Alcohol Dependence. 132 (1-2): 53-62).

### C. Xylazine

The U.S. Drug Enforcement Administration is warning the American public of a sharp increase in the trafficking of fentanyl mixed with xylazine. Xylazine, also known as “Tranq,” is a powerful sedative that the U.S. Food and Drug Administration has approved for veterinary use. Because xylazine is not an opioid, naloxone (Narcan) does not reverse its effects. Xylazine and fentanyl drug mixtures place users at a higher risk of suffering a fatal drug poisoning. Accordingly, an FDA administrator recently explained: “Xylazine is making the deadliest drug threat our country has ever faced, fentanyl, even deadlier.”

### D. Oxytocin

Oxytocin is indicated and approved by the FDA for two specific time frames in the obstetric world: antepartum and postpartum. In the antepartum period, exogenous oxytocin is FDA-approved for strengthening uterine contractions with the aim of successful vaginal delivery of the fetus. There are three situations during the antepartum period in which oxytocin is indicated: (i) for mothers who have preeclampsia, maternal diabetes, premature rupture of the membranes; (ii) for mothers with inactive uteri that require stimulation to start labor; and (iii) for mothers with inevitable or incomplete abortions in their second trimester.

In regards to the postpartum period, oxytocin is FDA-approved when it is time to deliver the placenta during the third stage of labor and control postpartum hemorrhage. A former version of oxytocin in the United States included an intranasal formula to encourage postpartum milk ejection. Other non-FDA-approved indications for exogenous oxytocin include treatment of delayed orgasm, inducing sexual arousal, and treatment of autism. Oxytocin has long been known as a hormone that plays a role in social behaviors and bonding. Because women release oxytocin during sexual intercourse, it is thought to play a role in bonding. Autism is not known to be caused by lower levels of oxytocin when compared to non-autistic people; however, previous studies have shown that giving oxytocin to children with autism seems to spark social skills.

Oxytocin is an oligopeptide hormone that contains nine amino acyl residues, or in other words, a nonapeptide hormone. It is one of the two hormones stored and released from the posterior pituitary gland but created in the hypothalamus. It is specifically released from the paraventricular nucleus of the hypothalamus into the posterior pituitary gland for later use. This specific part of the posterior pituitary gland that stores oxytocin is called the pars nervosa, also known as the neural or posterior lobe. Most hormones create negative feedback loops after they are released, but oxytocin is one of the few that exhibit positive feedback loops, i.e., that the release of oxytocin leads to actions that stimulate even more of a release of oxytocin. This feedback contrasts with antidiuretic hormone (ADH), also known as vasopressin (the second and only other hormone stored and released from the posterior pituitary), which exhibits a negative feedback loop after release. Less of this hormone will be released after it exhibits its effect on the body.

Exogenous oxytocin causes the same response in the female reproductive system as that of endogenous oxytocin. Both types of oxytocin stimulate uterine contractions in the myometrium by causing G-protein coupled receptors to stimulate a rise in intracellular calcium in uterine myofibrils. Oxytocin receptor activation causes many signals that stimulate uterine contraction by increasing intracellular calcium levels, which is where positive feedback comes into play. When oxytocin is released, it stimulates uterine contractions, and these uterine contractions, in turn, cause more oxytocin to be released; this is what causes the increase in both the intensity and frequency of contractions and enables a mother to carry out vaginal delivery completely. The head of the fetus pushes against the cervix, the nerve impulses from this action travel to the mother's brain, which activates the posterior pituitary to secrete oxytocin. This oxytocin is then carried through the blood to the uterus to increase uterine contractions further, and the cycle continues until parturition. Not only does oxytocin stimulate uterine contractions, but it also causes contractions of the myoepithelial cells in the female breasts. This activity occurs in the alveolar ducts. Such contractions are what force milk from these ducts into even larger sinuses, which enable milk expulsion. Positive feedback is also relevant to this milk-ejection reflex. A baby attempting to latch on to his mother's breast signals oxytocin secretion into the blood in the same manner as vaginal delivery, except, instead of uterine contractions, milk is ejected from the breast. The oxytocin makes its way to the brain at the same time to increase more oxytocin secretion. Lastly, oxytocin also has both antidiuretic and vasodilatory effects, increasing cerebral, coronary, and even renal blood flow.

### E. Compositions

### Pharmaceutical Formulations

Disclosed herein is a pharmaceutical formulation comprising oxytocin and one or more pharmaceutically acceptable carriers. Disclosed herein is a pharmaceutical formulation comprising oxytocin, naloxone, and one or more pharmaceutically acceptable carriers. Disclosed herein is a pharmaceutical formulation comprising oxytocin, nalmefene, and one or more pharmaceutically acceptable carriers. Disclosed herein is a pharmaceutical formulation comprising oxytocin and one or more opioid receptor antagonists, and one or more pharmaceutically acceptable carriers. Disclosed herein is a pharmaceutical formulation comprising oxytocin, naloxone, and one or more opioid receptor antagonists, and one or more pharmaceutically acceptable carriers.

Disclosed herein is a pharmaceutical formulation comprising oxytocin, nalmefene, and one or more opioid receptor antagonists, and one or more pharmaceutically acceptable carriers.

Disclosed herein is a pharmaceutical formulation comprising oxytocin, one or more additional therapeutic agents, and one or more pharmaceutically acceptable carriers. Disclosed herein is a pharmaceutical formulation comprising oxytocin, naloxone, one or more additional therapeutic agents, and one or more pharmaceutically acceptable carriers. Disclosed herein is a pharmaceutical formulation comprising oxytocin, nalmefene, one or more additional therapeutic agents, and one or more pharmaceutically acceptable carriers. Disclosed herein is a pharmaceutical formulation comprising oxytocin, one or more opioid receptor antagonists, one or more additional therapeutic agents, and one or more pharmaceutically acceptable carriers. Disclosed herein is a pharmaceutical formulation comprising oxytocin, naloxone, one or more additional therapeutic agents, one or more opioid receptor antagonists, and one or more pharmaceutically acceptable carriers. Disclosed herein is a pharmaceutical formulation comprising oxytocin, nalmefene, one or more opioid receptor antagonists, one or more additional therapeutic agents, and one or more pharmaceutically acceptable carriers.

In an aspect, oxytocin can comprise a pharmaceutically acceptable salt including pharmaceutically acceptable acid addition salt. In an aspect, naloxone can comprise a pharmaceutically acceptable salt including pharmaceutically acceptable acid addition salt. In an aspect, nalmefene can comprise a pharmaceutically acceptable salt including pharmaceutically acceptable acid addition salt.

In an aspect, oxytocin can comprise an analogue. Oxytocyin analogues are known to the art and include, for example, carbetocin, WAY-267,474, barusiban, Antag III).

In an aspect, a disclosed pharmaceutically acceptable acid addition salt can be prepared from pharmaceutically acceptable non-toxic acids including inorganic and organic acids. In an aspect, representative acids can comprise acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfuric, tartaric, oxalic, p-toluenesulfonic, and any combination thereof. In an aspect, pharmaceutically acceptable salts can comprise those listed by Berge et al. (1977) J. Pharma. Sci. 66:1-19.

In an aspect, a disclosed acid addition salts can be obtained as the direct products of compound synthesis. In an aspect, a free base can be dissolved in a suitable solvent containing the appropriate acid and the salt isolated by evaporating the solvent or otherwise separating the salt and solvent.

In an aspect, naloxone is an opioid receptor antagonist that is approved for use by injection for the reversal of opioid overdose and for adjunct use in the treatment of septic shock. It is currently being used mainly in emergency departments and in ambulances by trained medical professionals. There have been efforts to expand its use by providing the drug to some patients with take-home opioid prescriptions and those who inject illicit drugs, potentially facilitating earlier administration of the drug. The UN Commission on Narcotics Drugs “encourages all Member States to include effective elements for the prevention and treatment of drug overdose, in particular opioid overdose, in national drug policies, where appropriate, and to share best practices and information on the prevention and treatment of drug overdose, in particular opioid overdose, including the use of opioid receptor antagonists such as naloxone.”

In an aspect, a disclosed pharmaceutical formulation comprising oxytocin can be administered to a subject in need thereof. In an aspect, a disclosed pharmaceutical formulation comprising oxytocin can be formulated for administration and/or can be administered via one or more routes. Such methods are well known to those skilled in the art and include, but are not limited to, the following routes: oral administration, transdermal administration, administration by inhalation, nasal administration, topical administration, in utero administration, intrahepatic administration, intravaginal administration, ophthalmic administration, intraaural administration, otic administration, intracerebral administration, rectal administration, sublingual administration, buccal administration, and parenteral administration, including injectable such as intravenous administration, intra-CSF administration, intra-arterial administration, intramuscular administration, and subcutaneous administration.

In an aspect, a disclosed pharmaceutical formulation comprising oxytocin can be formulated for intravenous, intramuscular, sublingual, subcutaneous, intranasal, transnasal, oral, rectal, or transdermal administration.

In an aspect, a therapeutically effective amount of oxytocin and/or a salt and/or solvate thereof can comprise at least about 1 IU to at least about 200 IU. In an aspect, a therapeutically effective amount of oxytocin and/or a salt and/or solvate thereof can comprise at least about 1 IU to at least about 10 IU, at least about 10 IU to at least about 20 IU, at least about 20 IU to at least about 30 IU, at least about 30 IU to at least about 40 IU, at least about 40 IU to at least about 50 IU, at least about 50 IU to at least about 60 IU, at least about 60 IU to at least about 70 IU, at least about 70 IU to at least about 80 IU, at least about 80 IU to at least about 90 IU, at least about 90 IU to at least about 100 IU, at least about 100 IU to at least about 110 IU, at least about 110 IU to at least about 120 IU, at least about 120 IU to at least about 130 IU, at least about 130 IU to at least about 140 IU, at least about 140 IU to at least about 150 IU, at least about 150 IU to at least about 160 IU, at least about 160 IU to at least about 170 IU, at least about 170 IU to at least about 180 IU, at least about 180 IU to at least about 190 IU, or at least about 190 IU to at least about 200 IU. In an aspect, a therapeutically effective amount of oxytocin and/or a salt and/or solvate thereof can comprise at least about 1 IU, at least about 2 IU, at least about 3 IU, at least about 4 IU, at least about 5 IU, at least about 6 IU, at least about 7 IU, at least about 8 IU, at least about 9 IU, at least about 10 IU, at least about 11 IU, at least about 12 IU, at least about 13 IU, at least about 14 IU, at least about 15 IU, at least about 16 IU, at least about 17 IU, at least about 18 IU, at least about 19 IU, or at least about 20 IU.

In an aspect, a disclosed pharmaceutical formulation can comprise between about 1% (w/v) and about 20% (w/v) of oxytocin and/or a salt and/or solvate thereof. In an aspect, a disclosed pharmaceutical formulation can comprise between at least about 1% (w/v), at least about 2% (w/v), at least about 3% (w/v), at least about 4% (w/v), at least about 5% (w/v), at least about 6% (w/v), at least about 7% (w/v), at least about 8% (w/v), at least about 9% (w/v), at least about 10% (w/v), at least about 11% (w/v), at least about 12% (w/v), at least about 13% (w/v), at least about 14% (w/v), at least about 15% (w/v), at least about 16% (w/v), at least about 17% (w/v), at least about 18% (w/v), at least about 19% (w/v), or at least about 20% (w/v) of oxytocin and/or a salt and/or solvate thereof. In an aspect, a disclosed pharmaceutical formulation can comprise comprises between at least about 1% (w/v) to at least about 5% (w/v), at least about 5% (w/v) to at least about 10% (w/v), at least about 10% (w/v) to at least about 15% (w/v), or at least about 15% (w/v) to at least about 20% (w/v) of oxytocin and/or a salt and/or solvate thereof.

In an aspect, a disclosed pharmaceutical formulation can comprise between about 0.1 mg to about 350 mg of oxytocin and/or a salt and/or solvate thereof.

In an aspect, a disclosed pharmaceutical formulation can comprise oxytocin and/or a salt and/or solvate thereof in a range of about 0.001 mg to about 5 mg per kilogram of the subject's body weight.

In an aspect, a disclosed pharmaceutical formulation can comprise oxytocin and/or a salt and/or solvate thereof in an amount of about 1 mg total, about 10 mg total, about 100 mg total, about 200 mg total, about 300 total mg, or about 350 total mg.

In an aspect, a disclosed pharmaceutical formulation comprising oxytocin can further comprise at least about 1 IU to at least about 200 IU of naloxone and/or a salt and/or solvate thereof. In an aspect, a disclosed pharmaceutical formulation comprising oxytocin can further comprise at least about 1 IU to at least about 10 IU, at least about 10 IU to at least about 20 IU, at least about 20 IU to at least about 30 IU, at least about 30 IU to at least about 40 IU, at least about 40 IU to at least about 50 IU, at least about 50 IU to at least about 60 IU, at least about 60 IU to at least about 70 IU, at least about 70 IU to at least about 80 IU, at least about 80 IU to at least about 90 IU, at least about 90 IU to at least about 100 IU, at least about 100 IU to at least about 110 IU, at least about 110 IU to at least about 120 IU, at least about 120 IU to at least about 130 IU, at least about 130 IU to at least about 140 IU, at least about 140 IU to at least about 150 IU, at least about 150 IU to at least about 160 IU, at least about 160 IU to at least about 170 IU, at least about 170 IU to at least about 180 IU, at least about 180 IU to at least about 190 IU, or at least about 190 IU to at least about 200 IU of naloxone and/or a salt and/or solvate thereof. In an aspect, a disclosed pharmaceutical formulation comprising oxytocin can further comprise at least about 1 IU, at least about 2 IU, at least about 3 IU, at least about 4 IU, at least about 5 IU, at least about 6 IU, at least about 7 IU, at least about 8 IU, at least about 9 IU, at least about 10 IU, at least about 11 IU, at least about 12 IU, at least about 13 IU, at least about 14 IU, at least about 15 IU, at least about 16 IU, at least about 17 IU, at least about 18 IU, at least about 19 IU, or at least about 20 IU of naloxone and/or a salt and/or solvate thereof.

In an aspect, a disclosed pharmaceutical formulation comprising oxytocin can further comprise between about 1% (w/v) and about 20% (w/v) of naloxone and/or a salt and/or solvate thereof. In an aspect, a disclosed pharmaceutical formulation comprising oxytocin can further comprise between at least about 1% (w/v), at least about 2% (w/v), at least about 3% (w/v), at least about 4% (w/v), at least about 5% (w/v), at least about 6% (w/v), at least about 7% (w/v), at least about 8% (w/v), at least about 9% (w/v), at least about 10% (w/v), at least about 11% (w/v), at least about 12% (w/v), at least about 13% (w/v), at least about 14% (w/v), at least about 15% (w/v), at least about 16% (w/v), at least about 17% (w/v), at least about 18% (w/v), at least about 19% (w/v), or at least about 20% (w/v) of naloxone and/or a salt and/or solvate thereof.

In an aspect, a disclosed pharmaceutical formulation comprising oxytocin can further comprise between at least about 1% (w/v) to at least about 5% (w/v), at least about 5% (w/v) to at least about 10% (w/v), at least about 10% (w/v) to at least about 15% (w/v), or at least about 15% (w/v) to at least about 20% (w/v) of naloxone and/or a salt and/or solvate thereof.

For example, in an aspect, a disclosed naloxone can be naloxone hydrochloride dihydrate. In an aspect, a disclosed pharmaceutical formulation comprising oxytocin can further comprise at least about 1 IU to at least about 200 IU of nalmefene and/or a salt and/or solvate thereof. In an aspect, a disclosed pharmaceutical formulation comprising oxytocin can further comprise at least about 1 IU to at least about 10 IU, at least about 10 IU to at least about 20 IU, at least about 20 IU to at least about 30 IU, at least about 30 IU to at least about 40 IU, at least about 40 IU to at least about 50 IU, at least about 50 IU to at least about 60 IU, at least about 60 IU to at least about 70 IU, at least about 70 IU to at least about 80 IU, at least about 80 IU to at least about 90 IU, at least about 90 IU to at least about 100 IU, at least about 100 IU to at least about 110 IU, at least about 110 IU to at least about 120 IU, at least about 120 IU to at least about 130 IU, at least about 130 IU to at least about 140 IU, at least about 140 IU to at least about 150 IU, at least about 150 IU to at least about 160 IU, at least about 160 IU to at least about 170 IU, at least about 170 IU to at least about 180 IU, at least about 180 IU to at least about 190 IU, or at least about 190 IU to at least about 200 IU of nalmefene and/or a salt and/or solvate thereof. In an aspect, a disclosed pharmaceutical formulation comprising oxytocin can further comprise at least about 1 IU, at least about 2 IU, at least about 3 IU, at least about 4 IU, at least about 5 IU, at least about 6 IU, at least about 7 IU, at least about 8 IU, at least about 9 IU, at least about 10 IU, at least about 11 IU, at least about 12 IU, at least about 13 IU, at least about 14 IU, at least about 15 IU, at least about 16 IU, at least about 17 IU, at least about 18 IU, at least about 19 IU, or at least about 20 IU of nalmefene and/or a salt and/or solvate thereof.

In an aspect, a disclosed pharmaceutical formulation comprising oxytocin can further comprise between about 1% (w/v) and about 20% (w/v) of nalmefene and/or a salt and/or solvate thereof. In an aspect, a disclosed pharmaceutical formulation comprising oxytocin can further comprise between at least about 1% (w/v), at least about 2% (w/v), at least about 3% (w/v), at least about 4% (w/v), at least about 5% (w/v), at least about 6% (w/v), at least about 7% (w/v), at least about 8% (w/v), at least about 9% (w/v), at least about 10% (w/v), at least about 11% (w/v), at least about 12% (w/v), at least about 13% (w/v), at least about 14% (w/v), at least about 15% (w/v), at least about 16% (w/v), at least about 17% (w/v), at least about 18% (w/v), at least about 19% (w/v), or at least about 20% (w/v) of nalmefene and/or a salt and/or solvate thereof. In an aspect, a disclosed pharmaceutical formulation comprising oxytocin can comprise between at least about 1% (w/v) to at least about 5% (w/v), at least about 5% (w/v) to at least about 10% (w/v), at least about 10% (w/v) to at least about 15% (w/v), or at least about 15% (w/v) to at least about 20% (w/v) of nalmefene and/or a salt and/or solvate thereof.

In an aspect, a disclosed pharmaceutical formulation can comprise between at least about 1 mg and at least about 50 mg of oxytocin and/or a salt and/or solvate thereof. In an aspect, a disclosed pharmaceutical formulation can comprise at least about 1 mg, at least about 2 mg, at least about 3 mg, at least about 4 mg, at least about 5 mg, at least about 6 mg, at least about 7 mg, at least about 8 mg, at least about 9 mg, at least about 10 mg, at least about 11 mg, at least about 12 mg, at least about 13 mg, at least about 14 mg, at least about 15 mg, at least about 16 mg, at least about 17 mg, at least about 18 mg, at least about 19 mg, at least about 20 mg, at least about 21 mg, at least about 22 mg, at least about 23 mg, at least about 24 mg, at least about 25 mg, at least about 26 mg, at least about 27 mg, at least about 28 mg, at least about 29 mg, at least about 30 mg, at least about 31 mg, at least about 32 mg, at least about 33 mg, at least about 34 mg, at least about 35 mg, at least about 36 mg, at least about 37 mg, at least about 38 mg, at least about 39 mg, at least about 40 mg, at least about 41 mg, at least about 42 mg, at least about 43 mg, at least about 44 mg, at least about 45 mg, at least about 46 mg, at least about 47 mg, at least about 48 mg, at least about 49 mg, or at least about 50 mg of oxytocin and/or a salt and/or solvate thereof. In an aspect, a disclosed pharmaceutical formulation can comprise at least about 1 mg to least about 5 mg, at least about 5 mg to at least about 10 mg, at least about 10 mg to at least about 15 mg, at least about 15 mg to at least about 20 mg, at least about 20 mg to at least about 25 mg, at least about 25 mg to at least about 30 mg, at least about 30 mg to at least about 35 mg, at least about 35 mg to at least about 40 mg, at least about 40 mg to at least about 45 mg, or at least about 45 mg to at least about 50 mg of oxytocin and/or a salt and/or solvate thereof.

In an aspect, a disclosed pharmaceutical formulation comprising oxytocin can further comprise between at least about 1 mg and at least about 50 mg of naloxone and/or a salt and/or solvate thereof. I In an aspect, a disclosed pharmaceutical formulation comprising oxytocin can further comprise at least about 1 mg, at least about 2 mg, at least about 3 mg, at least about 4 mg, at least about 5 mg, at least about 6 mg, at least about 7 mg, at least about 8 mg, at least about 9 mg, at least about 10 mg, at least about 11 mg, at least about 12 mg, at least about 13 mg, at least about 14 mg, at least about 15 mg, at least about 16 mg, at least about 17 mg, at least about 18 mg, at least about 19 mg, at least about 20 mg, at least about 21 mg, at least about 22 mg, at least about 23 mg, at least about 24 mg, at least about 25 mg, at least about 26 mg, at least about 27 mg, at least about 28 mg, at least about 29 mg, at least about 30 mg, at least about 31 mg, at least about 32 mg, at least about 33 mg, at least about 34 mg, at least about 35 mg, at least about 36 mg, at least about 37 mg, at least about 38 mg, at least about 39 mg, at least about 40 mg, at least about 41 mg, at least about 42 mg, at least about 43 mg, at least about 44 mg, at least about 45 mg, at least about 46 mg, at least about 47 mg, at least about 48 mg, at least about 49 mg, or at least about 50 mg of naloxone and/or a salt and/or solvate thereof.

In an aspect, a disclosed pharmaceutical formulation comprising oxytocin can further comprise at least about 1 mg to least about 5 mg, at least about 5 mg to at least about 10 mg, at least about 10 mg to at least about 15 mg, at least about 15 mg to at least about 20 mg, at least about 20 mg to at least about 25 mg, at least about 25 mg to at least about 30 mg, at least about 30 mg to at least about 35 mg, at least about 35 mg to at least about 40 mg, at least about 40 mg to at least about 45 mg, or at least about 45 mg to at least about 50 mg of naloxone and/or a salt and/or solvate thereof. In an aspect, a disclosed pharmaceutical formulation comprising oxytocin can further comprise between at least about 1 mg and at least about 50 mg of nalmefene and/or a salt and/or solvate thereof. I In an aspect, a disclosed pharmaceutical formulation comprising oxytocin can further comprise at least about 1 mg, at least about 2 mg, at least about 3 mg, at least about 4 mg, at least about 5 mg, at least about 6 mg, at least about 7 mg, at least about 8 mg, at least about 9 mg, at least about 10 mg, at least about 11 mg, at least about 12 mg, at least about 13 mg, at least about 14 mg, at least about 15 mg, at least about 16 mg, at least about 17 mg, at least about 18 mg, at least about 19 mg, at least about 20 mg, at least about 21 mg, at least about 22 mg, at least about 23 mg, at least about 24 mg, at least about 25 mg, at least about 26 mg, at least about 27 mg, at least about 28 mg, at least about 29 mg, at least about 30 mg, at least about 31 mg, at least about 32 mg, at least about 33 mg, at least about 34 mg, at least about 35 mg, at least about 36 mg, at least about 37 mg, at least about 38 mg, at least about 39 mg, at least about 40 mg, at least about 41 mg, at least about 42 mg, at least about 43 mg, at least about 44 mg, at least about 45 mg, at least about 46 mg, at least about 47 mg, at least about 48 mg, at least about 49 mg, or at least about 50 mg of nalmefene and/or a salt and/or solvate thereof.

In an aspect, a disclosed pharmaceutical formulation comprising oxytocin can further comprise n an aspect, a disclosed pharmaceutical formulation can comprise at least about 1 mg to least about 5 mg, at least about 5 mg to at least about 10 mg, at least about 10 mg to at least about 15 mg, at least about 15 mg to at least about 20 mg, at least about 20 mg to at least about 25 mg, at least about 25 mg to at least about 30 mg, at least about 30 mg to at least about 35 mg, at least about 35 mg to at least about 40 mg, at least about 40 mg to at least about 45 mg, or at least about 45 mg to at least about 50 mg of nalmefene and/or a salt and/or solvate thereof. In an aspect, a therapeutically effective amount of a disclosed pharmaceutical formulation can comprise at least about 25 nmol/kg, at least about 50 nmol/kg, at least about 75 nmol/kg, at least about 100 nmol/kg, at least about 125 nmol/kg, at least about 150 nmol/kg, at least about 175 nmol/kg, at least about 200 nmol/kg, at least about 225 nmol/kg, at least about 250 nmol/kg, at least about 275 nmol/kg, at least about 300 nmol/kg, at least about 325 nmol/kg, at least about 350 nmol/kg, at least about 375 nmol/kg, at least about 400 nmol/kg, at least about 425 nmol/kg, at least about 450 nmol/kg, at least about 475 nmol/kg, or at least about 500 nmol/kg of oxytocin and/or a salt and/or solvate thereof.

In an aspect, a disclosed pharmaceutical formulation can be an aqueous solution. In an aspect, a dose of a disclosed aqueous solution can comprise between about 50 μL and about 500 μL. In an aspect, a dose of a disclosed aqueous solution can comprise between about 50 μL, about 100 μL, about 150 μL, about 200 μL, about 250 μL, about 300 μL, about 350 μL, about 400 μL, about 450 μL, about 500 μL, about 550 μL, about 600 μL, about 650 μL, about 700 μL, about 750 μL, about 800 μL, about 850 μL, about 900 μL, about 950 μL, or about 1000 μL.

In an aspect, a disclosed pharmaceutical formulation comprising oxytocin can further comprise one or more of an isotonicity agent, an acid, a preservative, a stabilizing agent, an absorption enhancer, and any combination thereof.

In an aspect, a disclosed pharmaceutical formulation comprising oxytocin can further comprise one or more tonicity agents. In an aspect, a disclosed tonicity agent can comprise dextrose, lactose, sodium chloride, calcium chloride, magnesium chloride, sorbitol, sucrose, mannitol, trehalose, raffinose, polyethylene glycol, hydroxyethyl starch, glycine, or any combination thereof. In an aspect, a disclosed tonicity agent can comprise sodium chloride. In an aspect, a disclosed pharmaceutical formulation comprising oxytocin can further comprise about 0.2% (w/v) and about 1.2% (w/v) of a tonicity agent. In an aspect, a disclosed pharmaceutical formulation comprising oxytocin can further comprise about 0.2% (w/v), about 0.3% (w/v), about 0.4% (w/v), about 0.5% (w/v), about 0.6% (w/v), about 0.7% (w/v), about 0.8% (w/v), about 0.9% (w/v), about 1.0% (w/v), about 1.1% (w/v), or about 1.2% (w/v) of a tonicity agent. In an aspect, a disclosed pharmaceutical formulation comprising oxytocin can further comprise more than about 0.2% (w/v) of a tonicity agent. In an aspect, a disclosed pharmaceutical formulation comprising oxytocin can further comprise less than about 1.2% (w/v) of a tonicity agent. In an aspect, a disclosed pharmaceutical formulation comprising oxytocin can further comprise about 0.1 mg and about 6 mg of a tonicity agent. In an aspect, a disclosed pharmaceutical formulation comprising oxytocin can further comprise about 0.2 mg, about 0.3 mg, about 0.4 mg, about 0.5 mg, about 0.6 mg, about 0.7 mg, about 0.8 mg, about 0.9 mg, about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg, about 2.1 mg, about 2.2 mg, about 2.3 mg, about 2.4 mg, about 2.5 mg, about 2.6 mg, about 2.7 mg, about 2.8 mg, about 2.9 mg, about 3.0 mg, about 3.1 mg, about 3.2 mg, about 3.3 mg, about 3.4 mg, about 3.5 mg, about 3.6 mg, about 3.7 mg, about 3.8 mg, about 3.9 mg, about 4.0 mg, about 4.1 mg, about 4.2 mg, about 4.3 mg, about 4.4 mg, about 4.5 mg, about 4.6 mg, about 4.7 mg, about 4.8 mg, about 4.9 mg, about 5.0 mg, about 5.1 mg, about 5.2 mg, about 5.3 mg, about 5.4 mg, about 5.5 mg, about 5.6 mg, about 5.7 mg, about 5.8 mg, about 5.9 mg, or about 6.0 mg of a tonicity agent.

In an aspect, a disclosed pharmaceutical formulation comprising oxytocin can further comprise one or more absorption enhancers. In an aspect, a disclosed pharmaceutical formulation comprising oxytocin can further comprise absorption enhancer can be an alkylglycoside. In an aspect, alkylsaccharides are used in commercial food and personal care products and have been designated Generally Recognized as Safe (GRAS) substances for food applications. They are non-irritating enhancers of transmucosal absorption that are odorless, tasteless, non-toxic, non-mutagenic, and non-sensitizing in the Draize test up to a 25% concentration. Alkylsaccharides can increase absorption by increasing paracellular permeability, as indicated by a decrease in transepithelial electrical resistance; they may also increase. The effect can be short-lived.

In an aspect, a disclosed alkylsaccharide absorption enhancer can comprise dodecyl maltoside, tetradecyl maltoside, octyl maltoside, decyl maltoside, nonyl maltoside, undecyl maltoside, tridecyl maltoside, pentadecyl maltoside, hexadecyl maltoside, heptadecyl maltoside, octadecyl-α-maltoside, octadecyl-β-D-maltoside, dodecyl glucoside, tetradecyl glucoside, octyl glucoside, decyl glucoside, nonyl glucoside, undecyl glucoside, tridecyl glucoside, pentadecyl glucoside, hexadecyl glucoside, heptadecyl glucoside, octadecyl-α-glucoside, and octadecyl-β-D-glucoside, dodecyl sucroside, tetradecyl sucroside, octyl sucroside, decyl sucroside, nonyl sucroside, undecyl sucroside, tridecyl sucroside, pentadecyl sucroside, hexadecyl sucroside, heptadecyl sucroside, octadecyl-α-sucroside, octadecyl-β-D-sucroside, or any combination thereof.

In an aspect, “saccharide” is inclusive of monosaccharides, oligosaccharides or polysaccharides in straight chain or ring forms, or a combination thereof to form a saccharide chain. In an aspect, oligosaccharides are saccharides having two or more monosaccharide residues. The saccharide can be chosen, for example, from any currently commercially available saccharide species or can be synthesized. In an aspect, some examples of the many possible saccharides to use include glucose, maltose, maltotriose, maltotetraose, sucrose, and trehalose.

In an aspect, a disclosed absorption enhancer can comprise aprotinin, benzalkonium chloride, benzyl alcohol, capric acid, ceramides, cetylpyridinium chloride, chitosan, cyclodextrins, deoxycholic acid, decanoyl carnitine, EDTA, glycocholic acid, glycodeoxycholic acid, glycofurol, glycosylated sphingosines, glycyrrhetinic acids, 2-hydroxypropyl-β-cyclodextrin, laureth-9, lauric acid, lauroyl carnitine, lauryl sulfate, lysophosphatidylcholine, menthol, poloxamer 407, poloxamer F68, poly-L-arginine, polyoxyethylene-9-lauryl ether, polysorbate 80, propylene glycol, quillaia saponin, salicylic acid, β-sitosterol-β-D-glucoside, sucrose cocoate, taurocholic acid, taurodeoxycholic acid, taurodihydrofusidic acid, and alkylsaccharides, such as dodecyl maltoside, tetradecyl maltoside, sucrose dodecanoate, or any combination thereof.

In an aspect, a disclosed alkylsaccharide can be Intravail® (dodecyl maltoside). As known to the art, Intravail® is the alkyl saccharide 1-O-n-dodecyl-β-D-maltopyranoside (alternately referred to as lauryl-β-D-maltopyranoside, dodecyl maltopyranoside, and DDM; C24H66O11). In an aspect, a disclosed absorption enhancer can be Intravail®.

In an aspect, a disclosed pharmaceutical formulation comprising oxytocin can further comprise between about 0.05% and about 2.5% (w/v), between about 0.1% and about 0.5% (w/v), between about 0.15% and about 0.35% (w/v), between about 0.15% and about 0.2% (w/v), or about 0.2% to about 0.3% (w/v) Intravail®.

In an aspect, a disclosed absorption enhancer can comprise benzalkonium chloride.

In an aspect, a disclosed absorption enhancer can comprise between about 0.005% (w/v) to about 0.05% (w/v), between about 0.005% to about 3.0% of the absorption enhancer, between about 0.05% to about 3.0% of the absorption enhancer, or between about 0.1% to about 0.5% of the absorption enhancer.

In an aspect, a disclosed pharmaceutical formulation comprising oxytocin can further comprise between about 0.1% to about 0.5%, about 0.5% to about 1.0%, about 1.0% to about 1.5%, about 1.5% to about 2.0%, about 2.0% to about 2.5%, about 2.5% to about 3.0%, about 3.0% to about 3.5%, about 3.5% to about 4.0%, about 4.0% to about 4.5%, or to about 4.5% to about 5.0% of the absorption enhancer.

In an aspect, a disclosed absorption enhancer can comprise benzalkonium chloride, chitosan, cyclodextrins, deoxycholic acid, dodecyl maltoside, tetradecyl maltoside, glycocholic acid, laureth-9, taurocholic acid, taurodihydrofusidic acid, or any combination thereof. In an aspect, the alkylsaccharide can comprise dodecyl maltoside, tetradecyl maltoside (TDM), and sucrose dodecanoate. In an aspect, a disclosed absorption enhancer can comprise about 0.05% (w/v) to about 2.5% (w/v) dodecyl maltoside or tetradecyl maltoside. In an aspect, a disclosed absorption enhancer can comprise about 0.05% (w/v) to about 0.25% (w/v), about 0.25% (w/v) to about 0.50% (w/v), about 0.50% (w/v) to about 0.75% (w/v), about 0.75% (w/v) to about 1.0% (w/v), about 1.0% (w/v) to about 1.25% (w/v), about 1.25% (w/v) to about 1.5% (w/v), about 1.5% (w/v) to about 1.75% (w/v), about 1.75% (w/v) to 2.0% (w/v), about 2.0% (w/v) to about 2.25% (w/v), or about 2.25% (w/v) to about 2.5% (w/v) dodecyl maltoside or tetradecyl maltoside.

In an aspect, a disclosed pharmaceutical formulation comprising oxytocin can further comprise one or more stabilizing agents. In an aspect, a disclosed stabilizing agent can comprise disodium edetate (EDTA). In an aspect, a disclosed stabilizing agent can be disodium edetate or edetate disodium.

In an aspect, a disclosed pharmaceutical formulation comprising oxytocin can further comprise one or more acids. In an aspect, a disclosed acid can be hydrochloric acid.

In an aspect, a disclosed pharmaceutical formulation comprising oxytocin can further comprise one or more preservatives. In an aspect, a disclosed preservative can comprise benzalkonium chloride, methylparaben, sodium benzoate, benzoic acid, phenyl ethyl alcohol, or any combination thereof.

In an aspect, a disclosed pharmaceutical formulation comprising oxytocin can further comprise one or more surfactants.

In an aspect, a disclosed surfactant can comprise polysorbate 80 NF, polyoxyethylene 20 sorbitan monolaurate, poly oxy ethylene (4) sorbitan monolaurate, poly oxy ethylene 20 sorbitan monopalmitate, poly oxy ethylene 20 sorbitan monostearate, poly oxy ethylene (4) sorbitan monostearate, poly oxy ethylene 20 sorbitan tristearate, poly oxy ethylene (5) sorbitan monooleate, polyoxyethylene 20 sorbitan trioleate, polyoxyethylene 20 sorbitan monoisostearate, sorbitan monooleate, sorbitan monolaurate, sorbitan monopalmitate, sorbitan monostearate, sorbitan trilaurate, sorbitan trioleate, sorbitan tristearate, and the like, and mixtures thereof.

In an aspect, a disclosed pharmaceutical formulation comprising oxytocin can further comprise one or more suspending agents. In an aspect, a disclosed suspending agent can comprise microcrystalline cellulose, carboxymethylcellulose sodium NF, polyacrylic acid, magnesium aluminum silicate, xanthan gum, or any combination thereof.

In an aspect, a disclosed pharmaceutical formulation comprising oxytocin can further comprise water. In an aspect, a disclosed pharmaceutical formulation comprising oxytocin can further comprise an aqueous solution. In an aspect, a disclosed aqueous solution can comprise about 10 μL, about 20 μL, about 30 μL, about 40 μL, about 50 μL, about 60 μL, about 70 μL, about 80 μL, about 90 μL, about 100 μL, about 100 μL, about 110 μL, about 120 μL, about 130 μL, about 140 μL, about 150 μL, about 160 μL, about 170 μL, about 180 μL, about 190 μL, about 200 μL, about 210 μL, about 220 μL, about 230 μL, about 240 μL, or about 250 μL.

In an aspect, a disclosed pharmaceutical formulation comprising oxytocin can further comprise water in an amount sufficient to achieve a final volume of at least about 20 μL, at least about 30 μL, at least about 40 μL, at least about 50 μL, at least about 60 μL, at least about 70 μL, at least about 80 μL, at least about 90 μL, at least about 100 μL, at least about 110 μL, at least about 120 μL, at least about 130 μL, at least about 140 μL, at least about 150 μL, at least about 160 μL, at least about 170 μL, at least about 180 μL, at least about 190 μL, at least about 200 μL, at least about 210 μL, at least about 220 μL, at least about 230 μL, at least about 240 μL, or at least about 250 μL.

In an aspect, water can be in an amount sufficient to total at least about 50 μL, at least about 100 μL, at least about 150 μL, at least about 200 μL, or at least about 250 μL.

Disclosed herein is a pharmaceutical formulation, comprising about 2% (w/v) to about 20% (w/v) oxytocin and/or a salt and/or solvate thereof; between about 0.2% (w/v) to about 1.2% (w/v) of an isotonicity agent; between about 0.005% (w/v) to about 0.05% (w/v) of a preservative; between about 0.1% (w/v) to about 0.5% (w/v) of a stabilizing agent; between about 0.1% (w/v) to about 0.5% (w/v) of an alkylsaccharide absorption enhancer; and water; wherein the pH of the formulation is between about 3.5 and about 5.5.

Disclosed herein is a pharmaceutical formulation, comprising about 2% (w/v) to about 20% (w/v) oxytocin and/or a salt and/or solvate thereof; about 2% (w/v) to about 20% (w/v) naloxone and/or a salt and/or solvate thereof; between about 0.2% (w/v) to about 1.2% (w/v) of an isotonicity agent; between about 0.005% (w/v) to about 0.05% (w/v) of a preservative; between about 0.1% (w/v) to about 0.5% (w/v) of a stabilizing agent; between about 0.1% (w/v) to about 0.5% (w/v) of an alkylsaccharide absorption enhancer; and water; wherein the pH of the formulation is between about 3.5 and about 5.5.

Disclosed herein is a pharmaceutical formulation, comprising about 2% (w/v) to about 20% (w/v) oxytocin and/or a salt and/or solvate thereof; about 2% (w/v) to about 20% (w/v) nalmefene and/or a salt and/or solvate thereof; between about 0.2% (w/v) to about 1.2% (w/v) of an isotonicity agent; between about 0.005% (w/v) to about 0.05% (w/v) of a preservative; between about 0.1% (w/v) to about 0.5% (w/v) of a stabilizing agent; between about 0.1% (w/v) to about 0.5% (w/v) of an alkylsaccharide absorption enhancer; and water; wherein the pH of the formulation is between about 3.5 and about 5.5.

Disclosed herein is a pharmaceutical formulation, comprising about 2% (w/v) to about 20% (w/v) oxytocin and/or a salt and/or solvate thereof; about 2% (w/v) to about 20% (w/v) naloxone and/or a salt and/or solvate thereof; about 2% (w/v) to about 20% (w/v) nalmefene and/or a salt and/or solvate thereof; between about 0.2% (w/v) to about 1.2% (w/v) of an isotonicity agent; between about 0.005% (w/v) to about 0.05% (w/v) of a preservative; between about 0.1% (w/v) to about 0.5% (w/v) of a stabilizing agent; between about 0.1% (w/v) to about 0.5% (w/v) of an alkylsaccharide absorption enhancer; and water; wherein the pH of the formulation is between about 3.5 and about 5.5.

Disclosed herein is a pharmaceutical formulation, comprising at least about 1 mg to at least about 10 mg oxytocin and/or a salt and/or solvate thereof; between about 0.2 mg and about 1.2 mg of an isotonicity agent; between about 0.005 mg and about 0.015 mg of preservative; between about 0.005 mg and about 0.015 mg of a cationic surfactant; between about 0.005 mg and about 0.015 mg of a permeation enhancer; between about 0.1 mg and about 0.5 mg of a stabilizing agent; and an amount of an acid sufficient to achieve a pH of 3.5-pH 5.5.

Disclosed herein is a pharmaceutical formulation, comprising at least about 1 mg to at least about 10 mg oxytocin and/or a salt and/or solvate thereof, at least about 1 mg to at least about 10 mg naloxone and/or a salt and/or solvate thereof; between about 0.2 mg and about 1.2 mg of an isotonicity agent; between about 0.005 mg and about 0.015 mg of preservative; between about 0.005 mg and about 0.015 mg of a cationic surfactant; between about 0.005 mg and about 0.015 mg of a permeation enhancer; between about 0.1 mg and about 0.5 mg of a stabilizing agent; and an amount of an acid sufficient to achieve a pH of 3.5-pH 5.5.

Disclosed herein is a pharmaceutical formulation, comprising at least about 1 mg to at least about 10 mg oxytocin and/or a salt and/or solvate thereof; at least about 1 mg to at least about 10 mg nalmefene and/or a salt and/or solvate thereof; between about 0.2 mg and about 1.2 mg of an isotonicity agent; between about 0.005 mg and about 0.015 mg of preservative; between about 0.005 mg and about 0.015 mg of a cationic surfactant; between about 0.005 mg and about 0.015 mg of a permeation enhancer; between about 0.1 mg and about 0.5 mg of a stabilizing agent; and an amount of an acid sufficient to achieve a pH of 3.5-pH 5.5.

Disclosed herein is a pharmaceutical formulation, comprising at least about 1 mg to at least about 10 mg oxytocin and/or a salt and/or solvate thereof; at least about 1 mg to at least about 10 mg nalmefene and/or a salt and/or solvate thereof; between about 0.2 mg and about 1.2 mg of an isotonicity agent; between about 0.005 mg and about 0.015 mg of preservative; between about 0.005 mg and about 0.015 mg of a cationic surfactant; between about 0.005 mg and about 0.015 mg of a permeation enhancer; between about 0.1 mg and about 0.5 mg of a stabilizing agent; and an amount of an acid sufficient to achieve a pH of 3.5-pH 5.5.

In an aspect, a therapeutically effective amount of a disclosed pharmaceutical formulation comprising oxytocin can yield, when intranasally administered to a subject, an oxytocin plasma concentration of at least about ≥0.2 ng/mL within 2.5 minutes in the subject; or an oxytocin plasma concentration of at least about ≥1 ng/ml within 5 minutes in the subject; or an oxytocin plasma concentration of at least about ≥3 ng/ml within 10 minutes in the subject.

In an aspect, a therapeutically effective amount of a disclosed pharmaceutical formulation comprising oxytocin can yield, when intranasally administered to a subject, an oxytocin Tₘₐₓ of less than about 30 minutes, less than about 25 minutes, less than about 20 minutes, or less than about 15 minutes.

In an aspect, a therapeutically effective amount of a disclosed pharmaceutical formulation comprising oxytocin can yield, when intranasally administered to a subject, a naloxone plasma concentration of at least about ≥0.2 ng/ml within 2.5 minutes in the subject; or a naloxone plasma concentration of at least about ≥1 ng/ml within 5 minutes in the subject; or a naloxone plasma concentration of at least about ≥3 ng/ml within 10 minutes in the subject.

In an aspect, a therapeutically effective amount of a disclosed pharmaceutical formulation comprising oxytocin can yield, when intranasally administered to a subject, a naloxone Tₘₐₓ of less than about 30 minutes, less than about 25 minutes, less than about 20 minutes, or less than about 15 minutes.

In an aspect, a therapeutically effective amount of a disclosed pharmaceutical formulation comprising oxytocin can yield, when intranasally administered to a subject, a nalmefene plasma concentration of at least about >0.2 ng/ml within 2.5 minutes in the subject; or a nalmefene plasma concentration of at least about >1 ng/ml within 5 minutes in the subject; or a nalmefene plasma concentration of at least about ≥3 ng/ml within 10 minutes in the subject.

In an aspect, a therapeutically effective amount of a disclosed pharmaceutical formulation comprising oxytocin can yield, when intranasally administered to a subject, a nalmefene Tₘₐₓ of less than about 30 minutes, less than about 25 minutes, less than about 20 minutes, or less than about 15 minutes.

In an aspect, a therapeutically effective amount of a disclosed pharmaceutical formulation comprising oxytocin can yield, when intranasally administered to a patient, (i) an oxytocin Tₘₐₓ of less than about 30 minutes, less than about 25 minutes, less than about 20 minutes, or less than about 15 minutes; (ii) a naloxone Tₘₐₓ of less than about 30 minutes, less than about 25 minutes, less than about 20 minutes, or less than about 15 minutes; (iii) a nalmefene oxytocin Tₘₐₓ of less than about 30 minutes, less than about 25 minutes, less than about 20 minutes, or less than about 15 minutes; or (iv) any combination thereof.

In an aspect, a therapeutically effective amount of a disclosed pharmaceutical formulation comprising oxytocin can yield, when intranasally administered to a subject, the plasma concentration versus time curve of oxytocin in the subject has a Tₘₐₓ of between about 20 and about 30 minutes; wherein the plasma concentration versus time curve of naloxone in the subject has a Tₘₐₓ of between about 20 and about 30 minutes; wherein the plasma concentration versus time curve of nalmefene in the subject has a Tₘₐₓ of between about 20 and about 30 minutes; or any combination thereof.

In an aspect, a disclosed pharmaceutical formulation comprising oxytocin can be used to treat a subject experiencing an opioid overdose due to fentanyl mixed with and/or laced with and/or cut with xylazine. In an aspect, a disclosed pharmaceutical formulation comprising oxytocin can be used to treat a subject suspected of experiencing an opioid overdose due to fentanyl mixed with and/or laced with and/or cut with xylazine.

In an aspect, a disclosed pharmaceutical formulation comprising oxytocin can be used to treat an opioid overdose or a suspected opioid overdose due to fentanyl mixed with and/or laced with and/or cut with xylazine.

In an aspect, a disclosed pharmaceutical formulation comprising oxytocin can be used to treat a subject experiencing opioid-induced respiratory depression (OIRD) due to fentanyl mixed with and/or laced with and/or cut with xylazine.

In an aspect, a disclosed pharmaceutical formulation comprising oxytocin can be used to treat a subject suspected of experiencing opioid-induced respiratory depression (OIRD) due to fentanyl mixed with and/or laced with and/or cut with xylazine.

In an aspect, a disclosed pharmaceutical formulation comprising oxytocin can be used to reduce and/or minimize and/or eliminate manifestations of anger, distress, aggression, and/or violence demonstrated by the subject following administration.

In an aspect, a disclosed pharmaceutical formulation comprising oxytocin can be used to treat a subject having an opioid dependence.

In an aspect, a disclosed pharmaceutical formulation comprising oxytocin can be used to treat a subject experiencing a depression of respiratory rate, a depression of minute ventilation, a depression of alveolar-arterial gas exchange, a decrease in respiratory responsiveness to hypoxia and hypercapnia, an increase in apnea duration and/or apnea frequency, an increase in hypoxemic events, cardiac arrest, and any combination thereof.

In an aspect, a disclosed pharmaceutical formulation comprising oxytocin can be used to treat a subject experiencing one or more symptoms associated with and/or related to an active opioid overdose due to fentanyl mixed with and/or laced with and/or cut with xylazine. In an aspect, a disclosed pharmaceutical formulation comprising oxytocin can be used to treat a subject experiencing one or more symptoms associated with and/or related to a suspected active opioid overdose due to fentanyl mixed with and/or laced with and/or cut with xylazine.

In an aspect, a disclosed pharmaceutical formulation comprising oxytocin can be used to treat a subject experiencing (i) a loss of consciousness, (ii) constricted or pinpoint pupils, (iii) breathing difficulties (slowed, labored, and/or irregular breathing), (iv) respiratory arrest, (v) choking, gurgling, or snoring sounds, (vi) blue or purple lips or fingertips, (vii) a low of responsive to loud noises, shaking, or painful stimuli, or (viii) any combination thereof.

In an aspect, a disclosed pharmaceutical formulation comprising oxytocin can be used to treat a subject experiencing one or more symptoms associated with and/or related to an active opioid overdose due to fentanyl mixed with and/or laced with and/or cut with xylazine, wherein the one or more symptoms associated with and/or related to the illicit use of opioids.

In an aspect, a disclosed pharmaceutical formulation comprising oxytocin can be used to treat a subject experiencing one or more symptoms associated with and/or related to an active opioid overdose, wherein the one or more symptoms associated with and/or related to the accidental misuse of opioids during medical opioid therapy.

In an aspect, a disclosed pharmaceutical formulation comprising oxytocin can be used to treat respiratory depression, central nervous system depression, cardiovascular depression, altered level consciousness, miotic pupils, hypoxemia, acute lung injury, aspiration pneumonia, sedation, hypotension, unresponsiveness to stimulus, unconsciousness, stopped breathing, erratic or stopped pulse, choking or gurgling sounds, blue or purple fingernails or lips, slack or limp muscle tone, contracted pupils, vomiting, and any combination thereof.

In an aspect, a disclosed pharmaceutical formulation comprising oxytocin can be used to minimize and/or reduce one or more symptoms of OIRD due to fentanyl mixed with and/or laced with and/or cut with xylazine. In an aspect, a disclosed pharmaceutical formulation comprising oxytocin can be used to reverse and/or eliminate one or more symptoms of OIRD due to fentanyl mixed with and/or laced with and/or cut with xylazine. In an aspect, a disclosed pharmaceutical formulation comprising oxytocin can be used to treat a subject having a substance use disorder.

In an aspect, a disclosed pharmaceutical formulation comprising oxytocin can be used to treat a subject having alcohol use disorder, cocaine use disorder, cannabis use disorder, tobacco use disorder, methamphetamine use disorder, opioid use disorder, or any combination thereof.

In an aspect, a disclosed pharmaceutical formulation comprising oxytocin can be used to treat a subject having a substance use disorder comprising the abuse of opioids.

In an aspect, opioids can comprise heroin, codeine, fentanyl, hydrocodone (dihydrocodeinone), hydromorphone, meperidine, methadone, morphine, oxycodone, oxymorphone, or any combination thereof. In an aspect, opioids can comprise fentanyl mixed with and/or laced with and/or cut with xylazine.

In an aspect, a disclosed pharmaceutical formulation comprising oxytocin can be used to treat a subject having at least one substance use withdrawal symptom. In an aspect, a disclosed pharmaceutical formulation comprising oxytocin can be used to treat a subject having at least one physical symptom of withdrawal, at least psychological symptom of withdrawal, or any combination thereof.

In an aspect, a disclosed physical symptom of withdrawal can comprise tremors, insomnia, disturbed sleep, headache, sweating, nausea, vomiting, muscle pain, muscle stiffness, hypertension, irregular heart rate, elevated heart rate, heart palpitations, dizziness, shakiness, tremors, seizures, dehydration, shallow breathing, fatigue, loss of appetite, clammy skin, loss of color, or anu combination thereof.

In an aspect, a disclosed pharmaceutical formulation comprising oxytocin can be used to reduce and/or eliminate at least one substance use withdrawal symptom in the subject.

In an aspect, a disclosed psychological symptom of withdrawal can comprise anxiety, irritability, difficulty concentrating, difficulty thinking clearly, mood swings, nightmares, depression, tension, panic attacks, short term memory loss, restlessness, a feeling of helplessness, stress-sensitivity, a heightened responsivity to substance-related cues, aberrant reward processing, a substance craving or a combination thereof. In an aspect, a disclosed pharmaceutical formulation comprising oxytocin can be used to reduce and/or eliminate at least one psychological symptom in the subject.

In an aspect, a disclosed pharmaceutical formulation comprising oxytocin can be formulated for intravenous, intramuscular, sublingual, subcutaneous, intranasal, transnasal, oral, rectal or transdermal administration.

In an aspect, a disclosed pharmaceutical formulation comprising oxytocin can further comprise one or more additional therapeutic agents. In an aspect, a disclosed pharmaceutical formulation comprising oxytocin can be used with one or more additional therapeutic agents.

In an aspect, a disclosed pharmaceutical formulation comprising can comprise (i) one or more active agents, (ii) biologically active agents, (iii) one or more pharmaceutically active agents, (iv) one or more immune-based therapeutic agents, (v) one or more clinically approved agents, or (vi) a combination thereof.

In an aspect, one or more disclosed pharmaceutical formulations comprising oxytocin can be subjected to one or more validating and/or characterizing steps and/or protocols. For example, in an aspect, validating and/or characterizing one or more disclosed pharmaceutical formulations comprising oxytocin can comprise measuring, ascertaining, and/or determining the purity and/or efficacy of the one or more disclosed pharmaceutical formulations.

In an aspect, a disclosed pharmaceutical formulation can improve and/or extend the survivability of the subject, can improve a subject's quality of life, can increase and/or prolong a subject's life span, or any combination thereof. In an aspect, patient life expectancy can be increased at least about 5% or greater to at least about 100%, at least about 10% or greater to at least about 95% or greater, at least about 20% or greater to at least about 80% or greater, at least about 40% or greater to at least about 60% or greater compared to an untreated subject with the identical or near identical overdose and the identical or near identical predicted outcome.

### Liquid Sprays

Disclosed herein is a liquid spray comprising oxytocin and one or more pharmaceutically acceptable carriers. Disclosed herein is a liquid spray comprising oxytocin, naloxone, and one or more pharmaceutically acceptable carriers. Disclosed herein is a liquid spray comprising oxytocin, nalmefene, and one or more pharmaceutically acceptable carriers. Disclosed herein is a liquid spray comprising oxytocin and one or more opioid receptor antagonists, and one or more pharmaceutically acceptable carriers.

Disclosed herein is a liquid spray comprising oxytocin, naloxone, and one or more opioid receptor antagonists, and one or more pharmaceutically acceptable carriers. Disclosed herein is a liquid spray comprising oxytocin, nalmefene, and one or more opioid receptor antagonists, and one or more pharmaceutically acceptable carriers. Disclosed herein is a liquid spray comprising oxytocin, one or more additional therapeutic agents, and one or more pharmaceutically acceptable carriers. Disclosed herein is a liquid spray comprising oxytocin, naloxone, one or more additional therapeutic agents, and one or more pharmaceutically acceptable carriers. Disclosed herein is a liquid spray comprising oxytocin, nalmefene, one or more additional therapeutic agents, and one or more pharmaceutically acceptable carriers.

Disclosed herein is a liquid spray comprising oxytocin, one or more opioid receptor antagonists, one or more additional therapeutic agents, and one or more pharmaceutically acceptable carriers. Disclosed herein is a liquid spray comprising oxytocin, naloxone, one or more additional therapeutic agents, one or more opioid receptor antagonists, and one or more pharmaceutically acceptable carriers. Disclosed herein is a liquid spray comprising oxytocin, nalmefene, one or more opioid receptor antagonists, one or more additional therapeutic agents, and one or more pharmaceutically acceptable carriers.

In an aspect, a disclosed liquid spray comprising oxytocin is for intranasal administration and/or transnasal administration.

In an aspect, a disclosed liquid spray comprising oxytocin does not contain sodium chloride, citric acid, benzyl alcohol, or benzalkonium chloride.

In an aspect, a disclosed liquid spray comprising oxytocin can comprise a co-solvent. In an aspect, a co-solvent can be alcohol and/or a glycol. In an aspect, a disclosed alcohol can be ethanol (dehydrated alcohol) and a disclosed glycol can be propylene glycol.

In an aspect, a disclosed liquid spray comprising oxytocin can have a pH from about 3.0 to about 6.0. In an aspect, a disclosed liquid sprays comprising oxytocin can have a pH of about 4.5.

In an aspect, a disclosed liquid spray comprising oxytocin can be used to treat a subject experiencing an opioid overdose due to fentanyl mixed with and/or laced with and/or cut with xylazine. In an aspect, a disclosed liquid spray comprising oxytocin can be used to treat a subject suspected of experiencing an opioid overdose due to fentanyl mixed with and/or laced with and/or cut with xylazine. In an aspect, a disclosed liquid spray comprising oxytocin can be used to treat an opioid overdose or a suspected opioid overdose due to fentanyl mixed with and/or laced with and/or cut with xylazine. In an aspect, a disclosed liquid spray comprising oxytocin can be used to treat a subject experiencing opioid-induced respiratory depression (OIRD) due to fentanyl mixed with and/or laced with and/or cut with xylazine. In an aspect, a disclosed liquid spray comprising oxytocin can be used to treat a subject suspected of experiencing opioid-induced respiratory depression (OIRD) due to fentanyl mixed with and/or laced with and/or cut with xylazine.

In an aspect, a disclosed liquid spray comprising oxytocin can be used to reduce and/or minimize and/or eliminate manifestations of anger, distress, aggression, and/or violence demonstrated by the subject following administration.

In an aspect, a disclosed liquid spray comprising oxytocin can be used to treat a subject having an opioid dependence.

In an aspect, a disclosed liquid spray comprising oxytocin can be used to treat a subject experiencing a depression of respiratory rate, a depression of minute ventilation, a depression of alveolar-arterial gas exchange, a decrease in respiratory responsiveness to hypoxia and hypercapnia, an increase in apnea duration and/or apnea frequency, an increase in hypoxemic events, cardiac arrest, and any combination thereof.

In an aspect, a disclosed liquid spray comprising oxytocin can be used to treat a subject experiencing one or more symptoms associated with and/or related to an active opioid overdose due to fentanyl mixed with and/or laced with and/or cut with xylazine. In an aspect, a disclosed liquid spray comprising oxytocin can be used to treat a subject experiencing one or more symptoms associated with and/or related to a suspected active opioid overdose due to fentanyl mixed with and/or laced with and/or cut with xylazine.

In an aspect, a disclosed liquid spray comprising oxytocin can be used to treat a subject experiencing (i) a loss of consciousness, (ii) constricted or pinpoint pupils, (iii) breathing difficulties (slowed, labored, and/or irregular breathing), (iv) respiratory arrest, (v) choking, gurgling, or snoring sounds, (vi) blue or purple lips or fingertips, (vii) a low of responsive to loud noises, shaking, or painful stimuli, or (viii) any combination thereof.

In an aspect, a disclosed liquid spray comprising oxytocin can be used to treat a subject experiencing one or more symptoms associated with and/or related to an active opioid overdose due to fentanyl mixed with and/or laced with and/or cut with xylazine, wherein the one or more symptoms associated with and/or related to the illicit use of opioids.

In an aspect, a disclosed liquid spray comprising oxytocin can be used to treat a subject experiencing one or more symptoms associated with and/or related to an active opioid overdose, wherein the one or more symptoms associated with and/or related to the accidental misuse of opioids during medical opioid therapy.

In an aspect, a disclosed liquid spray comprising oxytocin can be used to treat respiratory depression, central nervous system depression, cardiovascular depression, altered level consciousness, miotic pupils, hypoxemia, acute lung injury, aspiration pneumonia, sedation, hypotension, unresponsiveness to stimulus, unconsciousness, stopped breathing, erratic or stopped pulse, choking or gurgling sounds, blue or purple fingernails or lips, slack or limp muscle tone, contracted pupils, vomiting, and any combination thereof.

In an aspect, a disclosed liquid spray comprising oxytocin can be used to minimize and/or reduce one or more symptoms of OIRD due to fentanyl mixed with and/or laced with and/or cut with xylazine. In an aspect, a disclosed liquid spray comprising oxytocin can be used to reverse and/or eliminate one or more symptoms of OIRD due to fentanyl mixed with and/or laced with and/or cut with xylazine.

In an aspect, a disclosed liquid spray comprising oxytocin can be used to treat a subject having a substance use disorder.

In an aspect, a disclosed liquid spray comprising oxytocin can be used to treat a subject having alcohol use disorder, cocaine use disorder, cannabis use disorder, tobacco use disorder, methamphetamine use disorder, opioid use disorder, or any combination thereof.

In an aspect, a disclosed liquid spray comprising oxytocin can be used to treat a subject having a substance use disorder comprising the abuse of opioids.

In an aspect, opioids can comprise heroin, codeine, fentanyl, hydrocodone (dihydrocodeinone), hydromorphone, meperidine, methadone, morphine, oxycodone, oxymorphone, or any combination thereof. In an aspect, opioids can comprise fentanyl mixed with and/or laced with and/or cut with xylazine.

In an aspect, a disclosed liquid spray comprising oxytocin can be used to treat a subject having at least one substance use withdrawal symptom. In an aspect, a disclosed liquid spray comprising oxytocin can be used to treat a subject having at least one physical symptom of withdrawal, at least psychological symptom of withdrawal, or any combination thereof.

In an aspect, a disclosed physical symptom of withdrawal can comprise tremors, insomnia, disturbed sleep, headache, sweating, nausea, vomiting, muscle pain, muscle stiffness, hypertension, irregular heart rate, elevated heart rate, heart palpitations, dizziness, shakiness, tremors, seizures, dehydration, shallow breathing, fatigue, loss of appetite, clammy skin, loss of color, or anu combination thereof.

In an aspect, a disclosed liquid spray comprising oxytocin can be used to reduce and/or eliminate at least one substance use withdrawal symptom in the subject.

In an aspect, a disclosed psychological symptom of withdrawal can comprise anxiety, irritability, difficulty concentrating, difficulty thinking clearly, mood swings, nightmares, depression, tension, panic attacks, short term memory loss, restlessness, a feeling of helplessness, stress-sensitivity, a heightened responsivity to substance-related cues, aberrant reward processing, a substance craving or a combination thereof.

In an aspect, a disclosed liquid spray comprising oxytocin can be used to reduce and/or eliminate at least one psychological symptom in the subject. In an aspect, a disclosed liquid spray comprising oxytocin can be formulated for intravenous, intramuscular, sublingual, subcutaneous, intranasal, transnasal, oral, rectal, or transdermal administration.

In an aspect, a disclosed liquid spray can improve and/or extend the survivability of the subject, can improve a subject's quality of life, can increase and/or prolong a subject's life span, or any combination thereof. In an aspect, patient life expectancy can be increased at least about 5% or greater to at least about 100%, at least about 10% or greater to at least about 95% or greater, at least about 20% or greater to at least about 80% or greater, at least about 40% or greater to at least about 60% or greater compared to an untreated subject with the identical or near identical overdose and the identical or near identical predicted outcome.

### Devices

Disclosed herein are nasal drug delivery devices comprising a disclosed pharmaceutical formulation. Disclosed herein are nasal drug delivery devices comprising a disclosed liquid spray.

In an aspect, a disclosed device adapted for nasal delivery to a subject experiencing an opioid overdose. In an aspect, a disclosed device can be primed prior to use. In an aspect, a disclosed device can be actuated with one hand.

Nasal delivery is considered an attractive, safe, and easy-to-administer route for needle-free, systemic drug delivery, especially when rapid absorption and effect are desired. In addition, nasal delivery can help address issues related to poor bioavailability, slow absorption, drug degradation, and adverse events (AEs) in the gastrointestinal tract and avoids the first-pass metabolism in the liver.

In an aspect, a disclosed liquid nasal formulation can be an aqueous solution. In an aspect, a disclosed liquid nasal formulation can be a suspension or an emulsion. In an aspect, a disclosed liquid nasal formulation can comprise one or more antimicrobial preservatives.

In an aspect, a disclosed liquid nasal formulation can be delivered via a metered spray pump. In an aspect, a disclosed pump can deliver about 25 μL to about 200 μL per spray. In an aspect, a disclosed pump can deliver about 100 μL to about 150 μL per spray. In an aspect, a disclosed pump can off a high degree of reproducibility of an emitted dose. In an aspect, a disclosed pump can off a high degree of reproducibility of plume geometry. In an aspect, examples of standard metered spray pumps can include those offered by Aptar Pharma, Inc., such as the multi-dose “classic technology platform” nasal spray devices.

In an aspect, a disclosed device can comprise a reservoir that can hold multiple doses of a disclosed nasal spray formulation (e.g., 50, 100, 150, 200, 60, or 120 doses), a closure (e.g., screw, crimp, or snap-on), and an actuator which delivers anywhere from about 50 μL to about 1000 μL (e.g., about 50 μL, about 100 μL, about 150 μL, about 200 μL, about 250 μL, about 300 μL, about 350 μL, about 400 μL, about 450 μL, about 500 μL, about 550 μL, about 600 μL, about 650 μL, about 700 μL, about 750 μL, about 800 μL, about 850 μL, about 900 μL, about 950 μL, or about 1000 μL) of fluid per actuation to comprise a single dose.

In an aspect, a disclosed actuator can be configured to count doses, deliver gel formulations, deliver in an upside-down configuration, or any combination thereof. In an aspect, in traditional spray pump systems, antimicrobial preservatives can typically be required to maintain microbiological stability in liquid formulations. However, preservative-free systems are also available, e.g., the Advanced Preservative Free (APF) system from Aptar, which is vented, contains a filter membrane for air flow which prevents contamination, has a metal-free fluid path for oxidizing formulations, and can be used in any orientation. In an aspect, a disclosed device can be optimized with dispenser tips that prevent clogging (useful for high-viscosity and high-volatile formulations), actuators that do not need re-priming after long periods of disuse, etc.

In an aspect, particle size and plume geometry can vary within certain limits and depend on the properties of the pump, the formulation, the orifice of the actuator, and the force applied. In an aspect, droplet size can be influenced by the actuation parameters of the device and the formulation. In an aspect, a disclosed median droplet size can be between about 30 μm to about 100 μm. In an aspect, if the droplets are too large (>about 120 μm), then deposition can take place mainly in the anterior parts of the nose. In an aspect, if the droplets are too small (<about 10 μm), then the droplets can possibly be inhaled and reach the lungs, which should be avoided because of safety reasons. In an aspect, plume geometry, droplet size, and DSD of the delivered plume subsequent to spraying may be measured under specified experimental and instrumental conditions by appropriate and validated and/or calibrated analytical procedures known in the art (e.g., photography, laser diffraction, and impaction systems (cascade impaction, NGI)).

In an aspect, plume geometry, droplet size and DSD can affect pharmacokinetic outcomes such as Cₘₐₓ, Tₘₐₓ, and linear dose proportionality. In an aspect, droplet size distribution can be controlled in terms of ranges for the D10, D50, D90, span [(D90-D10)/D501, and percentage of droplets less than 10 mm. In an aspect, a disclosed liquid formulation can have a narrow DSD. In an aspect, a disclosed liquid formulation can have a D (v,50) of about 30 μm—about 70 μm and a D (v, 90)<100 μm.

In an aspect, the percent of droplets less than 10 μm can be less than 10%. In an aspect, the percent of droplets less than 10 μm can be less than 5%. In an aspect, the percent of droplets less than 10 μm can be less than 2%. In an aspect, the percent of droplets less than 10 μm can be less than 1%. In an aspect, a disclosed liquid formulation when dispensed by actuation from the device can produce a uniform circular plume with an ovality ratio close to 1. Ovality ratio is calculated as the quotient of the maximum diameter (Dₘₐₓ) and the minimum diameter (Dₘᵢₙ) of a spray pattern taken orthogonal to the direction of spray flow (e.g., from the “top”). In an aspect, a disclosed ovality ratio can be less than ±2.0. In an aspect, a disclosed ovality ratio can be less than ±1.5. In an aspect, a disclosed ovality ratio can be less than ±1.3. In an aspect, a disclosed ovality ratio can be less than ±1.2. In an aspect, a disclosed ovality ratio can be less than ±1.1. In an aspect, a disclosed ovality ratio can be about ±1.0. In an aspect, the 90% confidence interval for dose delivered per actuation can be±about 2%. In an aspect, the 95% confidence interval for dose delivered per actuation can be±about 2.5%.

In an aspect, intranasal administration of drugs in large volume, such as from syringes adapted with mucosal atomizer devices, has encountered difficulty due to the tendency of some of the formulation to drip back out of the nostril or down the nasopharynx.

In an aspect, a disclosed container closure system designs for inhalation spray drug products include both pre-metered and device-metered presentations using mechanical or power assistance and/or energy from patient inspiration for production of the spray plume. In an aspect, pre-metered presentations can contain previously measured doses or a dose fraction in some type of units (e.g., single or multiple blisters or other cavities) that are subsequently inserted into the device during manufacture or by the patient before use. In an aspect, typical device-metered units can have a reservoir containing formulation sufficient for multiple doses that are delivered as metered sprays by the device itself when activated by the patient.

In an aspect, a disclosed subject is an opioid overdose subject or a suspected opioid overdose subject.

In an aspect, a disclosed device comprising one or more disclosed pharmaceutical formulations comprising oxytocin and/or one or more disclosed liquid sprays comprising oxytocin can be used to treat a subject experiencing an opioid overdose due to fentanyl mixed with and/or laced with and/or cut with xylazine. In an aspect, a disclosed device comprising one or more disclosed pharmaceutical formulations comprising oxytocin and/or one or more disclosed liquid sprays comprising oxytocin can be used to treat a subject suspected of experiencing an opioid overdose due to fentanyl mixed with and/or laced with and/or cut with xylazine.

In an aspect, a disclosed device comprising one or more disclosed pharmaceutical formulations comprising oxytocin and/or one or more disclosed liquid sprays comprising oxytocin can be used to treat an opioid overdose or a suspected opioid overdose due to fentanyl mixed with and/or laced with and/or cut with xylazine.

In an aspect, a disclosed device comprising one or more disclosed pharmaceutical formulations comprising oxytocin and/or one or more disclosed liquid sprays comprising oxytocin can be used to treat a subject experiencing opioid-induced respiratory depression (OIRD) due to fentanyl mixed with and/or laced with and/or cut with xylazine.

In an aspect, a disclosed device comprising one or more disclosed pharmaceutical formulations comprising oxytocin and/or one or more disclosed liquid sprays comprising oxytocin can be used to treat a subject suspected of experiencing opioid-induced respiratory depression (OIRD) due to fentanyl mixed with and/or laced with and/or cut with xylazine.

In an aspect, a disclosed device comprising one or more disclosed pharmaceutical formulations comprising oxytocin and/or one or more disclosed liquid sprays comprising oxytocin can be used to reduce and/or minimize and/or eliminate manifestations of anger, distress, aggression, and/or violence demonstrated by the subject following administration.

In an aspect, a disclosed device comprising one or more disclosed pharmaceutical formulations comprising oxytocin and/or one or more disclosed liquid sprays comprising oxytocin can be used to treat a subject having an opioid dependence.

In an aspect, a disclosed device comprising one or more disclosed pharmaceutical formulations comprising oxytocin and/or one or more disclosed liquid sprays comprising oxytocin can be used to treat a subject experiencing a depression of respiratory rate, a depression of minute ventilation, a depression of alveolar-arterial gas exchange, a decrease in respiratory responsiveness to hypoxia and hypercapnia, an increase in apnea duration and/or apnea frequency, an increase in hypoxemic events, cardiac arrest, and any combination thereof.

In an aspect, a disclosed device comprising one or more disclosed pharmaceutical formulations comprising oxytocin and/or one or more disclosed liquid sprays comprising oxytocin can be used to treat a subject experiencing one or more symptoms associated with and/or related to an active opioid overdose due to fentanyl mixed with and/or laced with and/or cut with xylazine.

In an aspect, a disclosed device comprising one or more disclosed pharmaceutical formulations comprising oxytocin and/or one or more disclosed liquid sprays comprising oxytocin can be used to treat a subject experiencing one or more symptoms associated with and/or related to a suspected active opioid overdose due to fentanyl mixed with and/or laced with and/or cut with xylazine.

In an aspect, a disclosed device comprising one or more disclosed pharmaceutical formulations comprising oxytocin and/or one or more disclosed liquid sprays comprising oxytocin can be used to treat a subject experiencing (i) a loss of consciousness, (ii) constricted or pinpoint pupils, (iii) breathing difficulties (slowed, labored, and/or irregular breathing), (iv) respiratory arrest, (v) choking, gurgling, or snoring sounds, (vi) blue or purple lips or fingertips, (vii) a low of responsive to loud noises, shaking, or painful stimuli, or (viii) any combination thereof.

In an aspect, a disclosed device comprising one or more disclosed pharmaceutical formulations comprising oxytocin and/or one or more disclosed liquid sprays comprising oxytocin can be used to treat a subject experiencing one or more symptoms associated with and/or related to an active opioid overdose due to fentanyl mixed with and/or laced with and/or cut with xylazine, wherein the one or more symptoms associated with and/or related to the illicit use of opioids.

In an aspect, a disclosed device comprising one or more disclosed pharmaceutical formulations comprising oxytocin and/or one or more disclosed liquid sprays comprising oxytocin can be used to treat a subject experiencing one or more symptoms associated with and/or related to an active opioid overdose, wherein the one or more symptoms associated with and/or related to the accidental misuse of opioids during medical opioid therapy.

In an aspect, a disclosed device comprising one or more disclosed pharmaceutical formulations comprising oxytocin and/or one or more disclosed liquid sprays comprising oxytocin can be used to treat respiratory depression, central nervous system depression, cardiovascular depression, altered level consciousness, miotic pupils, hypoxemia, acute lung injury, aspiration pneumonia, sedation, hypotension, unresponsiveness to stimulus, unconsciousness, stopped breathing, erratic or stopped pulse, choking or gurgling sounds, blue or purple fingernails or lips, slack or limp muscle tone, contracted pupils, vomiting, and any combination thereof.

In an aspect, a disclosed device comprising one or more disclosed pharmaceutical formulations comprising oxytocin and/or one or more disclosed liquid sprays comprising oxytocin can be used to minimize and/or reduce one or more symptoms of OIRD due to fentanyl mixed with and/or laced with and/or cut with xylazine.

In an aspect, a disclosed device comprising one or more disclosed pharmaceutical formulations comprising oxytocin and/or one or more disclosed liquid sprays comprising oxytocin can be used to reverse and/or eliminate one or more symptoms of OIRD due to fentanyl mixed with and/or laced with and/or cut with xylazine.

In an aspect, a disclosed device comprising one or more disclosed pharmaceutical formulations comprising oxytocin and/or one or more disclosed liquid sprays comprising oxytocin can be used to treat a subject having a substance use disorder.

In an aspect, a disclosed device comprising one or more disclosed pharmaceutical formulations comprising oxytocin and/or one or more disclosed liquid sprays comprising oxytocin can be used to treat a subject having alcohol use disorder, cocaine use disorder, cannabis use disorder, tobacco use disorder, methamphetamine use disorder, opioid use disorder, or any combination thereof.

In an aspect, a disclosed device comprising one or more disclosed pharmaceutical formulations comprising oxytocin and/or one or more disclosed liquid sprays comprising oxytocin can be used to treat a subject having a substance use disorder comprising the abuse of opioids.

In an aspect, opioids can comprise heroin, codeine, fentanyl, hydrocodone (dihydrocodeinone), hydromorphone, meperidine, methadone, morphine, oxycodone, oxymorphone, or any combination thereof. In an aspect, opioids can comprise fentanyl mixed with and/or laced with and/or cut with xylazine.

In an aspect, a disclosed device comprising one or more disclosed pharmaceutical formulations comprising oxytocin and/or one or more disclosed liquid sprays comprising oxytocin can be used to treat a subject having at least one substance use withdrawal symptom. In an aspect, a disclosed device comprising one or more disclosed pharmaceutical formulations comprising oxytocin and/or one or more disclosed liquid sprays comprising oxytocin can be used to treat a subject having at least one physical symptom of withdrawal, at least psychological symptom of withdrawal, or any combination thereof.

In an aspect, a disclosed physical symptom of withdrawal can comprise tremors, insomnia, disturbed sleep, headache, sweating, nausea, vomiting, muscle pain, muscle stiffness, hypertension, irregular heart rate, elevated heart rate, heart palpitations, dizziness, shakiness, tremors, seizures, dehydration, shallow breathing, fatigue, loss of appetite, clammy skin, loss of color, or anu combination thereof.

In an aspect, a disclosed device comprising one or more disclosed pharmaceutical formulations comprising oxytocin and/or one or more disclosed liquid sprays comprising oxytocin can be used to reduce and/or eliminate at least one substance use withdrawal symptom in the subject.

In an aspect, a disclosed psychological symptom of withdrawal can comprise anxiety, irritability, difficulty concentrating, difficulty thinking clearly, mood swings, nightmares, depression, tension, panic attacks, short term memory loss, restlessness, a feeling of helplessness, stress-sensitivity, a heightened responsivity to substance-related cues, aberrant reward processing, a substance craving or a combination thereof.

In an aspect, a disclosed device comprising one or more disclosed pharmaceutical formulations comprising oxytocin and/or one or more disclosed liquid sprays comprising oxytocin can be used to reduce and/or eliminate at least one psychological symptom in the subject.

In an aspect, a disclosed device comprising one or more disclosed pharmaceutical formulations comprising oxytocin and/or one or more disclosed liquid sprays comprising oxytocin can be formulated for intravenous, intramuscular, sublingual, subcutaneous, intranasal, transnasal, oral, rectal, or transdermal administration.

In an aspect, a disclosed device can improve and/or extend the survivability of the subject, can improve a subject's quality of life, can increase and/or prolong a subject's life span, or any combination thereof. In an aspect, patient life expectancy can be increased at least about 5% or greater to at least about 100%, at least about 10% or greater to at least about 95% or greater, at least about 20% or greater to at least about 80% or greater, at least about 40% or greater to at least about 60% or greater compared to an untreated subject with the identical or near identical overdose and the identical or near identical predicted outcome.

### Kits

Disclosed herein is a kit comprising one or more disclosed pharmaceutical formulations comprising oxytocin, disclosed liquid sprays comprising oxytocin, disclosed devices for delivering oxytocin, or any combination thereof. Disclosed herein is a kit comprising one or more disclosed pharmaceutical formulations comprising oxytocin, disclosed liquid sprays comprising oxytocin, disclosed devices for delivering oxytocin, or any combination thereof and one or more additional therapeutic agents and/or pharmaceutical agents. Disclosed herein is a kit comprising one or more disclosed pharmaceutical formulations comprising oxytocin, disclosed liquid sprays comprising oxytocin, disclosed devices for delivering oxytocin, or any combination thereof for use in a method of treating a subject in active overdose due to fentanyl mixed with and/or laced with and/or cut with xylazine. Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating a subject suspected of having an active overdose due to fentanyl mixed with and/or laced with and/or cut with xylazine.

Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating a subject experiencing an opioid overdose due to fentanyl mixed with and/or laced with and/or cut with xylazine. Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating a subject suspected of experiencing an opioid overdose due to fentanyl mixed with and/or laced with and/or cut with xylazine.

Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating a subject experiencing opioid-induced respiratory depression (OIRD) due to fentanyl mixed with and/or laced with and/or cut with xylazine. Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating a subject suspected of experiencing opioid-induced respiratory depression (OIRD due to fentanyl mixed with and/or laced with and/or cut with xylazine.

Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating an opioid overdose due to fentanyl mixed with and/or laced with and/or cut with xylazine in a subject in need thereof. Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating a suspected opioid overdose due to fentanyl mixed with and/or laced with and/or cut with xylazine in a subject in need thereof.

Disclosed herein is a kit comprising one or more disclosed pharmaceutical formulations comprising oxytocin, disclosed liquid sprays comprising oxytocin, disclosed devices for delivery oxytocin, or any combination thereof for use in a method of reducing aggression, anger, and/or violence associated with reversal of an opioid overdose due to fentanyl mixed with and/or laced with and/or cut with xylazine in a subject in need thereof.

Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating a subject experiencing an opioid overdose due to fentanyl mixed with and/or laced with and/or cut with xylazine.

Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating a subject suspected of experiencing an opioid overdose due to fentanyl mixed with and/or laced with and/or cut with xylazine.

Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating a subject experiencing opioid-induced respiratory depression (OIRD) due to fentanyl mixed with and/or laced with and/or cut with xylazine. Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating subject suspected of experiencing opioid-induced respiratory depression (OIRD) due to fentanyl mixed with and/or laced with and/or cut with xylazine.

Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating an opioid overdose due to fentanyl mixed with and/or laced with and/or cut with xylazine in a subject in need thereof. Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating a suspected opioid overdose due to fentanyl mixed with and/or laced with and/or cut with xylazine in a subject in need thereof.

Disclosed herein is a kit comprising one or more disclosed pharmaceutical formulations comprising oxytocin, disclosed liquid sprays comprising oxytocin, disclosed devices for delivery oxytocin, or any combination thereof for use in a method of reducing aggression, anger, and/or violence associated with reversal of an opioid overdose due to fentanyl mixed with and/or laced with and/or cut with xylazine in a subject in need thereof.

Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating a subject experiencing an opioid overdose due to fentanyl mixed with and/or laced with and/or cut with xylazine.

Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating a subject suspected of experiencing an opioid overdose due to fentanyl mixed with and/or laced with and/or cut with xylazine.

Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating a subject experiencing opioid-induced respiratory depression (OIRD) due to fentanyl mixed with and/or laced with and/or cut with xylazine. Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating a subject suspected of experiencing opioid-induced respiratory depression (OIRD) due to fentanyl mixed with and/or laced with and/or cut with xylazine.

Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating an opioid overdose due to fentanyl mixed with and/or laced with and/or cut with xylazine in a subject in need thereof. Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating a suspected opioid overdose due to fentanyl mixed with and/or laced with and/or cut with xylazine in a subject in need thereof.

Disclosed herein is a kit comprising one or more disclosed pharmaceutical formulations comprising oxytocin, disclosed liquid sprays comprising oxytocin, disclosed devices for delivery oxytocin, or any combination thereof for use in a method of reducing aggression, anger, and/or violence associated with reversal of an opioid overdose due to fentanyl mixed with and/or laced with and/or cut with xylazine in a subject in need thereof.

Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating a subject experiencing an opioid overdose due to fentanyl mixed with and/or laced with and/or cut with xylazine. Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating a subject suspected of experiencing an opioid overdose due to fentanyl mixed with and/or laced with and/or cut with xylazine.

Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating a subject experiencing opioid-induced respiratory depression (OIRD) due to fentanyl mixed with and/or laced with and/or cut with xylazine. Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating a subject suspected of experiencing opioid-induced respiratory depression (OIRD) due to fentanyl mixed with and/or laced with and/or cut with xylazine.

Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating an opioid overdose due to fentanyl mixed with and/or laced with and/or cut with xylazine in a subject in need thereof. Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating a suspected opioid overdose due to fentanyl mixed with and/or laced with and/or cut with xylazine in a subject in need thereof.

Disclosed herein is a kit comprising one or more disclosed pharmaceutical formulations comprising oxytocin, disclosed liquid sprays comprising oxytocin, disclosed devices for delivery oxytocin, or any combination thereof for use in a method of reducing aggression, anger, and/or violence associated with reversal of an opioid overdose due to fentanyl mixed with and/or laced with and/or cut with xylazine in a subject in need thereof.

In an aspect, opioids can comprise fentanyl mixed with and/or laced with and/or cut with xylazine. In an aspect, a subject can be experiencing an opioid overdose due to fentanyl mixed with and/or laced with and/or cut with xylazine.

Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating a subject experiencing an opioid overdose due to fentanyl mixed with and/or laced with and/or cut with xylazine.

Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating a subject suspected of experiencing an opioid overdose due to fentanyl mixed with and/or laced with and/or cut with xylazine.

Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating a subject experiencing opioid-induced respiratory depression (OIRD) due to fentanyl mixed with and/or laced with and/or cut with xylazine a therapeutically effective amount of a disclosed pharmaceutical formulation.

Disclosed herein is a method of treating a subject, the method comprising administering to a subject suspected of experiencing opioid-induced respiratory depression (OIRD) due to fentanyl mixed with and/or laced with and/or cut with xylazine a therapeutically effective amount of a disclosed pharmaceutical formulation.

Disclosed herein is a method of treating an opioid overdose due to fentanyl mixed with and/or laced with and/or cut with xylazine, the method comprising administering to a subject in need thereof a therapeutically effective amount of a disclosed pharmaceutical formulation. Disclosed herein is a method of treating a suspected opioid overdose due to fentanyl mixed with and/or laced with and/or cut with xylazine, the method comprising administering to a subject in need thereof a therapeutically effective amount of a disclosed pharmaceutical formulation.

Disclosed herein is a method of reducing aggression, anger, and/or violence associated with reversal of an opioid overdose due to fentanyl mixed with and/or laced with and/or cut with xylazine, the method comprising administering to a subject in need thereof a therapeutically effective amount of a disclosed pharmaceutical formulation.

Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating a subject experiencing an opioid overdose due to fentanyl mixed with and/or laced with and/or cut with xylazine. Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating a subject suspected of experiencing an opioid overdose due to fentanyl mixed with and/or laced with and/or cut with xylazine.

Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating a subject experiencing opioid-induced respiratory depression (OIRD) due to fentanyl mixed with and/or laced with and/or cut with xylazine. Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating a subject suspected of experiencing opioid-induced respiratory depression (OIRD) due to fentanyl mixed with and/or laced with and/or cut with xylazine.

Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating an opioid overdose due to fentanyl mixed with and/or laced with and/or cut with xylazine in a subject in need thereof. Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating a suspected opioid overdose due to fentanyl mixed with and/or laced with and/or cut with xylazine in a subject in need thereof.

Disclosed herein is a kit comprising one or more disclosed pharmaceutical formulations comprising oxytocin, disclosed liquid sprays comprising oxytocin, disclosed devices for delivery oxytocin, or any combination thereof for use in a method of reducing aggression, anger, and/or violence associated with reversal of an opioid overdose due to fentanyl mixed with and/or laced with and/or cut with xylazine in a subject in need thereof.

Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating a subject experiencing an opioid overdose due to fentanyl mixed with and/or laced with and/or cut with xylazine.

Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating a subject suspected of experiencing an opioid overdose due to fentanyl mixed with and/or laced with and/or cut with xylazine.

Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating a subject experiencing opioid-induced respiratory depression (OIRD) due to fentanyl mixed with and/or laced with and/or cut with xylazine in a subject in need thereof. Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating a subject suspected of experiencing opioid-induced respiratory depression (OIRD) due to fentanyl mixed with and/or laced with and/or cut with xylazine in a subject in need thereof.

Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating an opioid overdose due to fentanyl mixed with and/or laced with and/or cut with xylazine in a subject in need thereof.

Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating a suspected opioid overdose due to fentanyl mixed with and/or laced with and/or cut with xylazine in a subject in need thereof. Disclosed herein is a kit comprising one or more disclosed pharmaceutical formulations comprising oxytocin, disclosed liquid sprays comprising oxytocin, disclosed devices for delivery oxytocin, or any combination thereof for use in a method of reducing aggression, anger, and/or violence associated with reversal of an opioid overdose due to fentanyl mixed with and/or laced with and/or cut with xylazine in a subject in need thereof.

Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating a subject experiencing an opioid overdose due to fentanyl mixed with and/or laced with and/or cut with xylazine. Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating a subject suspected of experiencing an opioid overdose due to fentanyl mixed with and/or laced with and/or cut with xylazine.

Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating a subject experiencing opioid-induced respiratory depression (OIRD) due to fentanyl mixed with and/or laced with and/or cut with xylazine. Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating a subject suspected of experiencing opioid-induced respiratory depression (OIRD) due to fentanyl mixed with and/or laced with and/or cut with xylazine.

Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating an opioid overdose due to fentanyl mixed with and/or laced with and/or cut with xylazine in a subject in need thereof.

Disclosed herein is a kit comprising one or more disclosed pharmaceutical formulations comprising oxytocin, disclosed liquid sprays comprising oxytocin, disclosed devices for delivery oxytocin, or any combination thereof for use in a method of treating a suspected opioid overdose due to fentanyl mixed with and/or laced with and/or cut with xylazine in a subject in need thereof.

Disclosed herein is a kit comprising one or more disclosed pharmaceutical formulations comprising oxytocin, disclosed liquid sprays comprising oxytocin, disclosed devices for delivery oxytocin, or any combination thereof for use in a method reducing aggression, anger, and/or violence associated with reversal of an opioid overdose due to fentanyl mixed with and/or laced with and/or cut with xylazine in a subject in need thereof.

In an aspect, by using a disclosed kit, an overdose due to fentanyl mixed with and/or laced with and/or cut with xylazine can be reversed.

In an aspect, a disclosed kit can be used to validate and/or characterize (i) one or more disclosed pharmaceutical formulations, and/or (ii) one or more disclosed or disclosed liquid sprays. In an aspect, validating and/or characterizing can comprise measuring, ascertaining, and/or determining the purity and/or efficacy of the one or more disclosed pharmaceutical formulations or one or more disclosed liquid sprays.

In an aspect, a disclosed kit can comprise at least two components constituting the kit. Together, the components constitute a functional unit for a given purpose (such as, for example, treating a subject experiencing an opioid overdose due to fentanyl mixed with and/or laced with and/or cut with xylazine or suspected of experiencing an opioid overdose due to fentanyl mixed with and/or laced with and/or cut with xylazine). Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

In an aspect, a disclosed kit for use in a disclosed method can comprise one or more containers holding one or more disclosed pharmaceutical formulations or one or more disclosed liquid sprays, disclosed opioid antagonists, disclosed pharmacological agents, or a combination thereof, and a label or package insert with instructions for use. In an aspect, suitable containers include, for example, bottles, vials, syringes, blister pack, etc. The containers can be formed from a variety of materials such as glass or plastic. The container can hold one or more disclosed pharmaceutical formulations or one or more disclosed liquid sprays, disclosed opioid antagonists, disclosed pharmacological agents, or a combination thereof, and can have a sterile access port (for example the container may be an intravenous solution bag or a vial having a stopper pierceable by a hypodermic injection needle). The label or package insert can indicate one or more disclosed pharmaceutical formulations or one or more disclosed liquid sprays, disclosed opioid antagonists, disclosed pharmacological agents, or a combination thereof can be used for use in a disclosed method. A kit can comprise additional components necessary for administration such as, for example, other buffers, diluents, filters, needles, and syringes.

In an aspect, a disclosed kit can improve and/or extend the survivability of the subject, can improve a subject's quality of life, can increase and/or prolong a subject's life span, or any combination thereof. In an aspect, patient life expectancy can be increased at least about 5% or greater to at least about 100%, at least about 10% or greater to at least about 95% or greater, at least about 20% or greater to at least about 80% or greater, at least about 40% or greater to at least about 60% or greater compared to an untreated subject with the identical or near identical overdose and the identical or near identical predicted outcome.

### Methods

Disclosed herein is a method of treating a subject, the method comprising administering to a subject experiencing an opioid overdose a therapeutically effective amount of a disclosed pharmaceutical formulation. Disclosed herein is a method of treating a subject, the method comprising administering to a subject suspected of experiencing an opioid overdose a therapeutically effective amount of a disclosed pharmaceutical formulation.

Disclosed herein is a method of treating a subject, the method comprising administering to a subject experiencing opioid-induced respiratory depression (OIRD) a therapeutically effective amount of a disclosed pharmaceutical formulation. Disclosed herein is a method of treating a subject, the method comprising administering to a subject suspected of experiencing opioid-induced respiratory depression (OIRD) a therapeutically effective amount of a disclosed pharmaceutical formulation.

Disclosed herein is a method of treating an opioid overdose, the method comprising administering to a subject in need thereof a therapeutically effective amount of a disclosed pharmaceutical formulation. Disclosed herein is a method of treating a suspected opioid overdose, the method comprising administering to a subject in need thereof a therapeutically effective amount of a disclosed pharmaceutical formulation.

Disclosed herein is a method of reducing aggression, anger, and/or violence associated with reversal of an opioid overdose, the method comprising administering to a subject in need thereof a therapeutically effective amount of a disclosed pharmaceutical formulation. Disclosed herein is a method of treating a subject, the method comprising administering to a subject experiencing an opioid overdose a therapeutically effective amount of a disclosed pharmaceutical formulation.

Disclosed herein is a method of treating a subject, the method comprising administering to a subject suspected of experiencing an opioid overdose a therapeutically effective amount of a disclosed pharmaceutical formulation.

Disclosed herein is a method of treating a subject, the method comprising administering to a subject experiencing opioid-induced respiratory depression (OIRD) a therapeutically effective amount of a disclosed pharmaceutical formulation. Disclosed herein is a method of treating a subject, the method comprising administering to a subject suspected of experiencing opioid-induced respiratory depression (OIRD) a therapeutically effective amount of a disclosed pharmaceutical formulation.

Disclosed herein is a method of treating an opioid overdose, the method comprising administering to a subject in need thereof a therapeutically effective amount of a disclosed pharmaceutical formulation. Disclosed herein is a method of treating a suspected opioid overdose, the method comprising administering to a subject in need thereof a therapeutically effective amount of a disclosed pharmaceutical formulation.

Disclosed herein is a method of reducing aggression, anger, and/or violence associated with reversal of an opioid overdose, the method comprising administering to a subject in need thereof a therapeutically effective amount of a disclosed pharmaceutical formulation.

Disclosed herein is a method of treating a subject, the method comprising administering to a subject experiencing an opioid overdose a therapeutically effective amount of a disclosed pharmaceutical formulation. Disclosed herein is a method of treating a subject, the method comprising administering to a subject suspected of experiencing an opioid overdose a therapeutically effective amount of a disclosed pharmaceutical formulation.

Disclosed herein is a method of treating a subject, the method comprising administering to a subject experiencing opioid-induced respiratory depression (OIRD) a therapeutically effective amount of a disclosed pharmaceutical formulation. Disclosed herein is a method of treating a subject, the method comprising administering to a subject suspected of experiencing opioid-induced respiratory depression (OIRD) a therapeutically effective amount of a disclosed pharmaceutical formulation.

Disclosed herein is a method of treating an opioid overdose, the method comprising administering to a subject in need thereof a therapeutically effective amount of a disclosed pharmaceutical formulation. Disclosed herein is a method of treating a suspected opioid overdose, the method comprising administering to a subject in need thereof a therapeutically effective amount of a disclosed pharmaceutical formulation.

Disclosed herein is a method of reducing aggression, anger, and/or violence associated with reversal of an opioid overdose, the method comprising administering to a subject in need thereof a therapeutically effective amount of a disclosed pharmaceutical formulation.

Disclosed herein is a method of treating a subject, the method comprising administering to a subject experiencing an opioid overdose a therapeutically effective amount of a disclosed pharmaceutical formulation.

Disclosed herein is a method of treating a subject, the method comprising administering to a subject suspected of experiencing an opioid overdose a therapeutically effective amount of a disclosed pharmaceutical formulation.

Disclosed herein is a method of treating a subject, the method comprising administering to a subject experiencing opioid-induced respiratory depression (OIRD) a therapeutically effective amount of a disclosed pharmaceutical formulation. Disclosed herein is a method of treating a subject, the method comprising administering to a subject suspected of experiencing opioid-induced respiratory depression (OIRD) a therapeutically effective amount of a disclosed pharmaceutical formulation.

Disclosed herein is a method of treating an opioid overdose, the method comprising administering to a subject in need thereof a therapeutically effective amount of a disclosed pharmaceutical formulation. Disclosed herein is a method of treating a suspected opioid overdose, the method comprising administering to a subject in need thereof a therapeutically effective amount a disclosed pharmaceutical formulation.

Disclosed herein is a method of reducing aggression, anger, and/or violence associated with reversal of an opioid overdose, the method comprising administering to a subject in need thereof a therapeutically effective amount of a disclosed pharmaceutical formulation.

In an aspect, opioids can comprise fentanyl mixed with and/or laced with and/or cut with xylazine. In an aspect, a subject can be experiencing an opioid overdose due to fentanyl mixed with and/or laced with and/or cut with xylazine.

Disclosed herein is a method of treating a subject, the method comprising administering to a subject experiencing an opioid overdose due to fentanyl mixed with and/or laced with and/or cut with xylazine a therapeutically effective amount of a disclosed pharmaceutical formulation.

Disclosed herein is a method of treating a subject, the method comprising administering to a subject suspected of experiencing an opioid overdose due to fentanyl mixed with and/or laced with and/or cut with xylazine a therapeutically effective amount of a disclosed pharmaceutical formulation.

Disclosed herein is a method of treating a subject, the method comprising administering to a subject experiencing opioid-induced respiratory depression (OIRD) due to fentanyl mixed with and/or laced with and/or cut with xylazine a therapeutically effective amount of a disclosed pharmaceutical formulation. Disclosed herein is a method of treating a subject, the method comprising administering to a subject suspected of experiencing opioid-induced respiratory depression (OIRD) due to fentanyl mixed with and/or laced with and/or cut with xylazine a therapeutically effective amount of a disclosed pharmaceutical formulation.

Disclosed herein is a method of treating an opioid overdose due to fentanyl mixed with and/or laced with and/or cut with xylazine, the method comprising administering to a subject in need thereof a therapeutically effective amount of a disclosed pharmaceutical formulation. Disclosed herein is a method of treating a suspected opioid overdose due to fentanyl mixed with and/or laced with and/or cut with xylazine, the method comprising administering to a subject in need thereof a therapeutically effective amount of a disclosed pharmaceutical formulation.

Disclosed herein is a method of reducing aggression, anger, and/or violence associated with reversal of an opioid overdose due to fentanyl mixed with and/or laced with and/or cut with xylazine, the method comprising administering to a subject in need thereof a therapeutically effective amount of a disclosed pharmaceutical formulation. Disclosed herein is a method of treating a subject, the method comprising administering to a subject experiencing an opioid overdose due to fentanyl mixed with and/or laced with and/or cut with xylazine a therapeutically effective amount of a disclosed pharmaceutical formulation.

Disclosed herein is a method of treating a subject, the method comprising administering to a subject suspected of experiencing an opioid overdose due to fentanyl mixed with and/or laced with and/or cut with xylazine a therapeutically effective amount of a disclosed pharmaceutical formulation.

Disclosed herein is a method of treating a subject, the method comprising administering to a subject experiencing opioid-induced respiratory depression (OIRD) due to fentanyl mixed with and/or laced with and/or cut with xylazine a therapeutically effective amount of a disclosed pharmaceutical formulation. Disclosed herein is a method of treating a subject, the method comprising administering to a subject suspected of experiencing opioid-induced respiratory depression (OIRD) due to fentanyl mixed with and/or laced with and/or cut with xylazine a therapeutically effective amount of a disclosed pharmaceutical formulation.

Disclosed herein is a method of treating an opioid overdose due to fentanyl mixed with and/or laced with and/or cut with xylazine, the method comprising administering to a subject in need thereof a therapeutically effective amount of a disclosed pharmaceutical formulation. Disclosed herein is a method of treating a suspected opioid overdose due to fentanyl mixed with and/or laced with and/or cut with xylazine, the method comprising administering to a subject in need thereof a therapeutically effective amount of a disclosed pharmaceutical formulation.

Disclosed herein is a method of reducing aggression, anger, and/or violence associated with reversal of an opioid overdose due to fentanyl mixed with and/or laced with and/or cut with xylazine, the method comprising administering to a subject in need thereof a therapeutically effective amount of a disclosed pharmaceutical formulation.

Disclosed herein is a method of treating a subject, the method comprising administering to a subject experiencing an opioid overdose due to fentanyl mixed with and/or laced with and/or cut with xylazine a therapeutically effective amount of a disclosed pharmaceutical formulation.

Disclosed herein is a method of treating a subject, the method comprising administering to a subject suspected of experiencing an opioid overdose due to fentanyl mixed with and/or laced with and/or cut with xylazine a therapeutically effective amount of a disclosed pharmaceutical formulation.

Disclosed herein is a method of treating a subject, the method comprising administering to a subject experiencing opioid-induced respiratory depression (OIRD) due to fentanyl mixed with and/or laced with and/or cut with xylazine a therapeutically effective amount of a disclosed pharmaceutical formulation. Disclosed herein is a method of treating a subject, the method comprising administering to a subject suspected of experiencing opioid-induced respiratory depression (OIRD) due to fentanyl mixed with and/or laced with and/or cut with xylazine a therapeutically effective amount of a disclosed pharmaceutical formulation.

Disclosed herein is a method of treating an opioid overdose due to fentanyl mixed with and/or laced with and/or cut with xylazine, the method comprising administering to a subject in need thereof a therapeutically effective amount of a disclosed pharmaceutical formulation. Disclosed herein is a method of treating a suspected opioid overdose due to fentanyl mixed with and/or laced with and/or cut with xylazine, the method comprising administering to a subject in need thereof a therapeutically effective amount of a disclosed pharmaceutical formulation.

Disclosed herein is a method of reducing aggression, anger, and/or violence associated with reversal of an opioid overdose due to fentanyl mixed with and/or laced with and/or cut with xylazine, the method comprising administering to a subject in need thereof a therapeutically effective amount of a disclosed pharmaceutical formulation.

Disclosed herein is a method of treating a subject, the method comprising administering to a subject experiencing an opioid overdose due to fentanyl mixed with and/or laced with and/or cut with xylazine a therapeutically effective amount of a disclosed pharmaceutical formulation. Disclosed herein is a method of treating a subject, the method comprising administering to a subject suspected of experiencing an opioid overdose due to fentanyl mixed with and/or laced with and/or cut with xylazine a therapeutically effective amount of a disclosed pharmaceutical formulation.

Disclosed herein is a method of treating a subject, the method comprising administering to a subject experiencing opioid-induced respiratory depression (OIRD) due to fentanyl mixed with and/or laced with and/or cut with xylazine a therapeutically effective amount of a disclosed pharmaceutical formulation. Disclosed herein is a method of treating a subject, the method comprising administering to a subject suspected of experiencing opioid-induced respiratory depression (OIRD) due to fentanyl mixed with and/or laced with and/or cut with xylazine a therapeutically effective amount of a disclosed pharmaceutical formulation.

Disclosed herein is a method of treating an opioid overdose due to fentanyl mixed with and/or laced with and/or cut with xylazine, the method comprising administering to a subject in need thereof a therapeutically effective amount of a disclosed pharmaceutical formulation. Disclosed herein is a method of treating a suspected opioid overdose due to fentanyl mixed with and/or laced with and/or cut with xylazine, the method comprising administering to a subject in need thereof a therapeutically effective amount a disclosed pharmaceutical formulation.

Disclosed herein is a method of reducing aggression, anger, and/or violence associated with reversal of an opioid overdose due to fentanyl mixed with and/or laced with and/or cut with xylazine, the method comprising administering to a subject in need thereof a therapeutically effective amount of a disclosed pharmaceutical formulation.

In an aspect, a disclosed method can further comprise repeating the administering of a disclosed pharmaceutical formulation. In an aspect of a disclosed method, administering can comprise intranasal administration, sublingual administration, intraperitoneal administration, intravenous administration, or a combination thereof.

In an aspect, a disclosed pharmaceutical formulation can be administered via the same routes of administration. In an aspect, a disclosed pharmaceutical formulation can be administered via the different routes of administration. In an aspect, administering can comprise using a disclosed device adapted for intranasal delivery of the pharmaceutical formulation. In an aspect, intranasal delivery can comprise one actuation of the device into one or both nostrils of the subject. In an aspect, intranasal delivery can comprise one actuation into one nostril or both nostrils of the subject using a disclosed device.

In an aspect of a disclosed method, a subject can be experiencing an active opioid overdose due to fentanyl mixed with and/or laced with and/or cut with xylazine. In an aspect of a disclosed method, a subject can be suspected of experiencing an active opioid overdose due to fentanyl mixed with and/or laced with and/or cut with xylazine.

In an aspect of a disclosed method, opioid-induced respiratory depression (OIRD) can comprise one or more of a depression of respiratory rate, a depression of minute ventilation, a depression of alveolar-arterial gas exchange, a decrease in respiratory responsiveness to hypoxia and hypercapnia, an increase in apnea duration and/or apnea frequency, an increase in hypoxemic events, cardiac arrest, and any combination thereof.

In an aspect of a disclosed method, following an administering step, one or more symptoms of opioid-induced respiratory depression (OIRD) can be improved and/or minimized. For example, in an aspect, following an administering step, a subject can experience an increase in respiratory rate, an increase in minute ventilation, an increase in alveolar-arterial gas exchange, an increase in respiratory responsiveness, a decrease in apnea duration and/or apnea frequency, an decrease in hypoxemic events, and any combination thereof.

In an aspect of a disclosed method, a subject can be experiencing one or more symptoms associated with and/or related to an active opioid overdose due to fentanyl mixed with and/or laced with and/or cut with xylazine.

In an aspect of a disclosed method, symptoms associated with and/or related to an active opioid overdose can comprise (i) a loss of consciousness, (ii) constricted or pinpoint pupils, (iii) breathing difficulties (slowed, labored, and/or irregular breathing), (iv) respiratory arrest, (v) choking, gurgling, or snoring sounds, (vi) blue or purple lips or fingertips, (vii) a low of responsive to loud noises, shaking, or painful stimuli, or (viii) any combination thereof.

In an aspect of a disclosed method, a subject can be exhibiting one or more symptoms associated with and/or related to the illicit use of opioids (e.g., fentanyl mixed with and/or laced with and/or cut with xylazine). In an aspect of a disclosed method, a subject can be exhibiting one or more symptoms associated with and/or related to the accidental misuse of opioids during medical opioid therapy.

In an aspect of a disclosed method, following the administering step, a subject can be free from respiratory depression for at least about 1 hour, at least about 2 hours, at least about 3 hours, at least about 4 hours, at least about 5 hours, or at least about 6 hours following.

In an aspect of a disclosed method, an administering step can be performed on a subject in a lying, supine, or recovery position. In an aspect of a disclosed method, one or more disclosed symptoms can comprise respiratory depression, central nervous system depression, cardiovascular depression, altered level consciousness, miotic pupils, hypoxemia, acute lung injury, aspiration pneumonia, sedation, hypotension, unresponsiveness to stimulus, unconsciousness, stopped breathing, erratic or stopped pulse, choking or gurgling sounds, blue or purple fingernails or lips, slack or limp muscle tone, contracted pupils, vomiting, and any combination thereof.

In an aspect of a disclosed method, following the administering step, one or more symptoms of OIRD due to fentanyl mixed with and/or laced with and/or cut with xylazine can be minimized and/or reduced. In an aspect of a disclosed method, following the administering step, one or more symptoms of OIRD due to fentanyl mixed with and/or laced with and/or cut with xylazine can be reversed and/or eliminated.

In an aspect of a disclosed method, the subject can have a substance use disorder.

In an aspect of a disclosed method, a disclosed substance use disorder can comprise alcohol use disorder, cocaine use disorder, cannabis use disorder, tobacco use disorder, methamphetamine use disorder, opioid use disorder, or any combination thereof. In an aspect of a disclosed method, a disclosed substance use disorder can comprise abuse of opioids. In an aspect, a disclosed opioid can comprise heroin, codeine, fentanyl, hydrocodone (dihydrocodeinone), hydromorphone, meperidine, methadone, morphine, oxycodone, oxymorphone, or any combination thereof.

In an aspect, a disclosed method can further comprise administering to the subject one or more additional pharmacotherapeutic agents for opioid use disorder. In an aspect, a disclosed method can further comprise administering to the subject one or more additional pharmacotherapies for substance abuse. In an aspect, one or more disclosed additional pharmacotherapeutic agents can comprise buprenorphine, methadone, naltrexone, or any combination thereof. In an aspect, one or more disclosed additional pharmacotherapeutic agents can comprise naloxone, nalmefene, or any combination thereof. In an aspect, disclosed additional pharmacotherapy can comprise a gradually reducing regimen, a substitution therapy, or a medication assisted therapy. In an aspect, a disclosed method can further comprise administering to the subject one or more additional therapies. In an aspect, one or more disclosed additional therapies can comprise a behavioral therapy.

In an aspect, behavioral therapy can comprise counseling, a contingency management system, a mindfulness-based therapy, a cognitive-behavioral therapy, a digitally administered behavioral therapy or a virtual reality based behavioral therapy, or any combination thereof. In an aspect, counseling can be in person or via digital means. In an aspect, a disclosed method can reduce the amount behavioral therapy required compared behavioral therapy without the administration of the composition.

In an aspect, a disclosed substance use disorder can comprise abuse of an opioid, and wherein the substitution therapy comprises Methadone or Buprenorphine. In an aspect, a disclosed substance use disorder can comprise abuse of an opioid, and wherein the medication assisted treatment comprises naltrexone, disulfiram, acamprosate, or any combination thereof.

In an aspect, a disclosed substance use withdrawal symptom can comprise a symptom of withdrawal from opioids. In an aspect, a disclosed opioid can comprise heroin, codeine, fentanyl, hydrocodone (dihydrocodeinone), hydromorphone, meperidine, methadone, morphine, oxycodone, oxymorphone, or any combination thereof. In an aspect, a disclosed opioid can comprise fentanyl mixed with and/or laced with and/or cut with xylazine.

In an aspect, a disclosed substance use withdrawal symptom can comprise a physical symptom of withdrawal, a psychological symptom of withdrawal, or any combination thereof.

In an aspect, a disclosed physical symptom of withdrawal can comprise tremors, insomnia, disturbed sleep, headache, sweating, nausea, vomiting, muscle pain, muscle stiffness, hypertension, irregular heart rate, elevated heart rate, heart palpitations, dizziness, shakiness, tremors, seizures, dehydration, shallow breathing, fatigue, loss of appetite, clammy skin, loss of color, or any combination thereof.

In an aspect, a disclosed psychological symptom of withdrawal can comprise anxiety, irritability, difficulty concentrating, difficulty thinking clearly, mood swings, nightmares, depression, tension, panic attacks, short term memory loss, restlessness, a feeling of helplessness, stress-sensitivity, a heightened responsivity to substance-related cues, aberrant reward processing, a substance craving or a combination thereof.

In an aspect, a disclosed method can further comprise an additional pharmacotherapy for withdrawal. In an aspect, a disclosed additional pharmacotherapy can comprise a gradually reducing regimen, a substitution therapy or a medication assisted treatment.

In an aspect, a disclosed method can further comprise a behavioral therapy. In an aspect, a disclosed behavioral therapy can comprise counseling, a contingency management system, a mindfulness-based therapy, a cognitive-behavioral therapy, a digitally administered behavioral therapy or a virtual reality based behavioral therapy.

In an aspect of a disclosed method, a disclosed pharmaceutical formulation can reduce or eliminate the at least one substance use withdrawal symptom in the subject. In an aspect of a disclosed method, a disclosed pharmaceutical formulation can reduce or eliminate the at least one psychological symptom in the subject.

In an aspect, a disclosed method can reduce a symptom of withdrawal or prevents a relapse of the substance use disorder in the subject. In an aspect, a disclosed method can reduce tolerance to a substance of the substance use disorder in the subject. In an aspect, a disclosed method can reduce dependence on a substance of the substance use disorder in the subject.

In an aspect, a disclosed method can improve adherence to a treatment for the substance use disorder in the subject. In an aspect, a disclosed method can reduce a preference for a substance or decreases liking for a substance. In an aspect, a disclosed method can increase abstinence from a substance.

In an aspect of a disclosed method, administering a disclosed pharmaceutical formulation can comprise a single dose or multiple doses (such as 2, 3, 4, 5, 6, 7, 8, 9, 10, or more than 10 doses). In an aspect of a disclosed method, administering one or more disclosed pharmaceutical formulations can comprise a single dose or multiple doses (such as 2, 3, 4, 5, 6, 7, 8, 9, 10, or more than 10 doses). In an aspect of a disclosed method, administering a disclosed pharmaceutical formulation can comprise a single dose or multiple doses (such as 2, 3, 4, 5, 6, 7, 8, 9, 10, or more than 10 doses).

In an aspect, a disclosed method can improve and/or extend the survivability of the subject, can improve a subject's quality of life, can increase and/or prolong a subject's life span, or any combination thereof. In an aspect, patient life expectancy can be increased at least about 5% or greater to at least about 100%, at least about 10% or greater to at least about 95% or greater, at least about 20% or greater to at least about 80% or greater, at least about 40% or greater to at least about 60% or greater compared to an untreated subject with the identical or near identical overdose and the identical or near identical predicted outcome.

In an aspect, a disclosed method can further comprise monitoring the subject for adverse effects. In an aspect, in the absence of adverse effects, a disclosed method can further comprise continuing to treat the subject. In an aspect, continuing to treat the subject can comprise continuing to administer a disclosed pharmaceutical formulation.

In an aspect, in the presence of adverse effects, a disclosed method can further comprise modifying one or more steps of the method. In an aspect, modifying one or more steps of a disclosed method can comprise modifying one or more administering steps. In an aspect, modifying one or more disclosed administering steps can comprise changing the amount of a disclosed composition (i.e., a disclosed pharmaceutical formulation or a disclosed liquid spray) administered to the subject, changing the frequency of a disclosed composition (i.e., a disclosed pharmaceutical formulation or a disclosed liquid spray) administration, changing the duration of a disclosed composition (i.e., a disclosed pharmaceutical formulation or a disclosed liquid spray) administration, changing the route of a disclosed composition (i.e., a disclosed pharmaceutical formulation or a disclosed liquid spray) administration, or any combination thereof. In an aspect, modifying one or more disclosed administering steps can comprise changing the amount a disclosed composition (i.e., a disclosed pharmaceutical formulation or a disclosed liquid spray) administered to the subject, changing the frequency of a disclosed composition (i.e., a disclosed pharmaceutical formulation or a disclosed liquid spray) administration, changing the duration of a disclosed composition (i.e., a disclosed pharmaceutical formulation or a disclosed liquid spray) administration, changing the route of a disclosed composition (i.e., a disclosed pharmaceutical formulation or a disclosed liquid spray), or any combination thereof.

In an aspect, a disclosed method can further comprise providing additional respiratory support to the subject. In an aspect, a disclosed method can further comprise providing additional respiratory support to the subject such as, for example, supplemental oxygen or one or more additional pharmacological agents.

In an aspect, a subject can be a human patient. In an aspect a subject can be any age (e.g., geriatric, adult, young adult, teenager, tween, adolescent, child, toddler, baby, or infant), can be male or female, can be any nationality, can be of any ethnicity, and/or can be of any race. In an aspect, a subject can be in active overdose. In an aspect, a subject can be suspected of having an active overdose. In an aspect, a subject can be in active overdose due to fentanyl mixed with and/or laced with and/or cut with xylazine. In an aspect, a subject can be suspected of having an active overdose due to fentanyl mixed with and/or laced with and/or cut with xylazine.

In an aspect, a disclosed method can comprise improving and/or enhancing the subject's quality of life. In an aspect of a disclosed method, a disclosed improvement and/or enhancement (such as, for example, in the subject's quality of life) can comprise a 10%, 20%, 30%, 40%, 50%, 60%, 70%, 80%, 90%, 100%, or any amount of improvement when compared to a control subject (such as, for example, a subject that has not received a disclosed pharmaceutical formulation).

In an aspect, a disclosed method can further comprise monitoring the subject following the administering step and/or the treating step to generate a compilation of biochemical and/or physiological and/or behavioral data. In an aspect, a disclosed compilation of data can be used to identify a trend or a pattern. In an aspect, a disclosed compilation of data can be used to guide and/or inform a skilled clinician in a decision-making process regarding treatment and/or testing. In an aspect, for example, a clinician can decide to change an aspect of the subject's treatment and/or change the subject's diagnosis or prognosis.

In an aspect, a disclosed method can further comprise monitoring the subject's metabolic and/or physiologic improvement following the administering and/or treating step and/or following the administering and/or treating steps. In an aspect, a clinician can measure and/or determine the subject's metabolic and/or physiologic status over time to identify one or more improvements and/or one or more diminishments. In an aspect of a disclosed method, a clinician can use the subject's metabolic and/or physiologic status and/or the trend of the subject's metabolic and/or physiological status and/or trend to make a treatment decision and/or to modify an aspect of a disclosed method and/or to continue treating the subject and/or continue to administer one or more disclosed pharmaceutical formulation thereof. In an aspect, metabolic and/or physiologic data can inform the clinician when make subsequent treatment decisions.

In an aspect, a disclosed method can further comprise repeating one or more disclosed steps of a disclosed method. In an aspect, a disclosed method can further comprise continuing to administer to the subject a therapeutically effective amount of one or more disclosed pharmaceutical formulations, one or more disclosed liquid sprays, one or more disclosed devices, or any combination thereof.

In an aspect, a disclosed method can further comprise generating and/or making one or more disclosed pharmaceutical formulations thereof. In an aspect, a disclosed method can further comprise validating and/or characterizing one or more disclosed pharmaceutical formulations thereof. For example, in an aspect, validating and/or characterizing one or more disclosed pharmaceutical formulations thereof can comprise measuring, ascertaining, and/or determining the purity and/or efficacy of the one or more disclosed pharmaceutical formulations thereof.

### VI. EXAMPLES

The Examples that follow are illustrative of specific aspects of the invention, and various uses thereof. The Examples set forth for explanatory purposes only and are not to be taken as limiting the invention.

The ability of fentanyl and xylazine to increase lethality is substantial, reflecting a synergistic (i.e., greater than additive) interaction. While fentanyl and xylazine each produce lethality characterized by steep dose-effect curves, fentanyl and xylazine significantly increase the lethal effects of one another in a synergistic manner. Moreover, lethality produced by the combination of fentanyl and xylazine is more rapid than high doses of fentanyl administered alone.

### Example 1

### Oxytocin Reversed Opioid-Induced Respiratory Depression

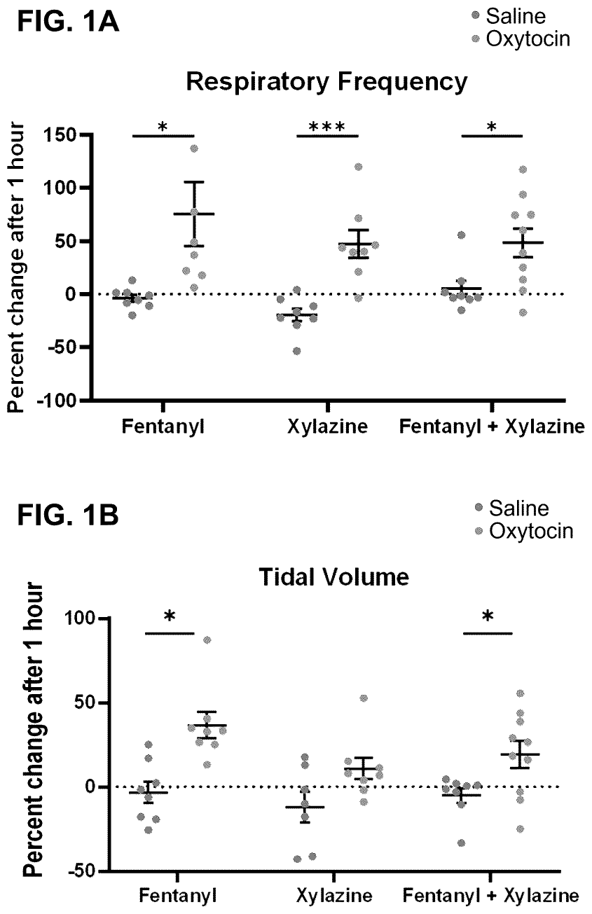

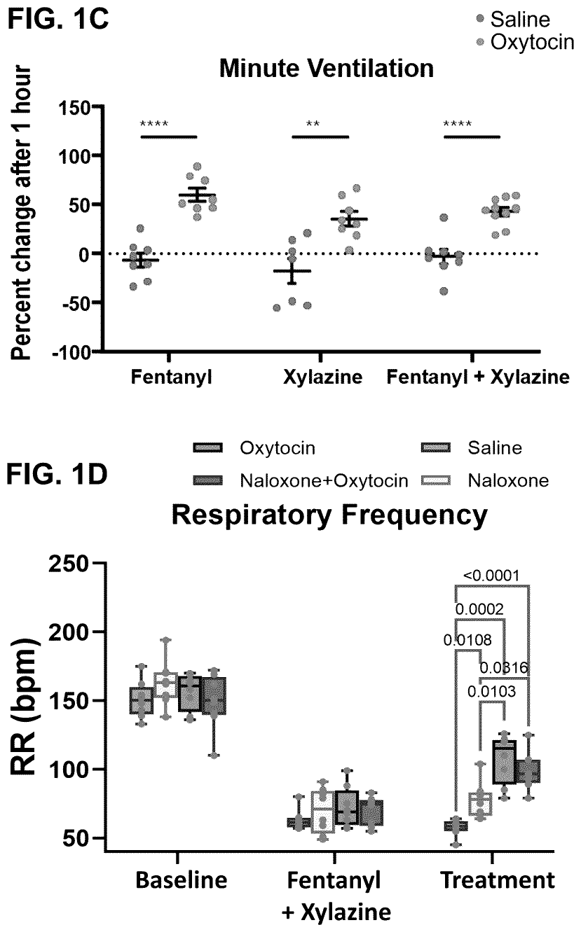

Three groups of male Sprague Dawley rats (n=8) were administered intraperitoneal (IP) fentanyl (0.5 mg/kg) and xylazine (1 mg/kg) either alone or in combination. Then, 10-15 minutes post-injection, xylazine, like fentanyl, significantly elicited respiratory depression. Oxytocin (200 nmol/kg) injected intraperitoneally 10-15 minutes after fentanyl significantly reversed OIRD and increased breathing frequency (FIG. 1A), tidal volume (FIG. 1B), and minute ventilation (FIG. 1C), compared to untreated controls. The combination of fentanyl+xylazine was a particularly potent mix that elicited respiratory depression. Naloxone was only partially effective in reversing opioid induced respiratory depression elicited by combined fentanyl+xylazine. Oxytocin potently reversed opioid induced respiratory depression elicited by combined fentanyl+xylazine (FIG. 1D).

### Example 2

### Oxytocin Reduced Occurrence of Apneas

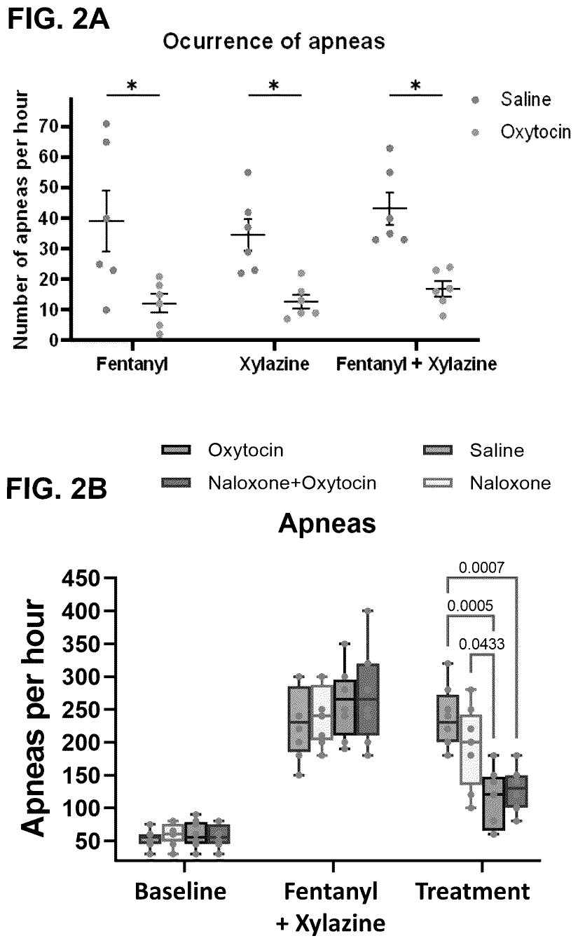

Oxytocin significantly diminished the incidence of apneas and hypopneas evoked by fentanyl (FIG. 2A). Oxytocin was significantly potent compared to naloxone in diminishing of the occurrence of apneas evoked by fentanyl (FIG. 2B). Respiratory function was quantified using a whole-body plethysmography system in unrestrained and freely moving animals.

### Example 3

### Oxytocin Increased Survival Following Opioid Overdose

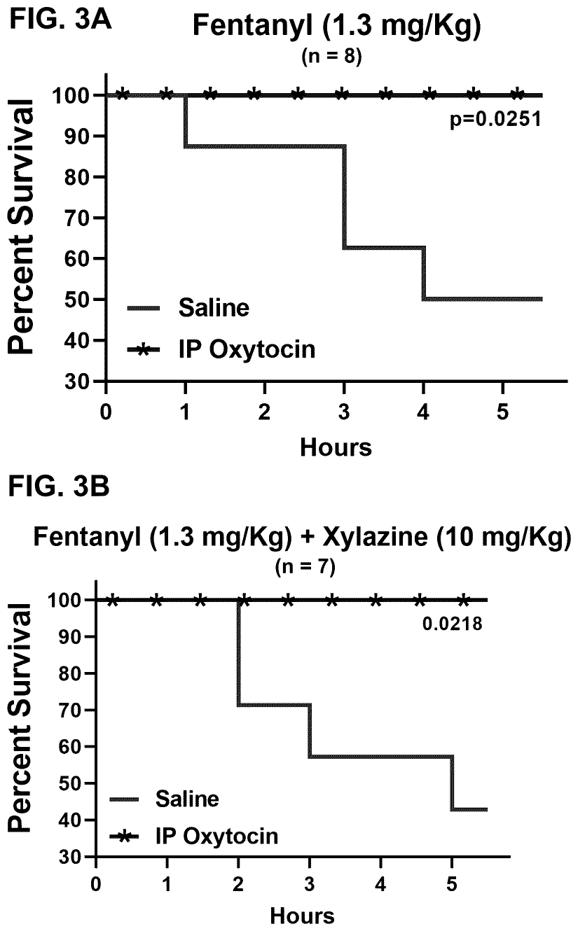

When using a lethal dose of fentanyl (1.3 mg/kg), the intraperitoneal injection of oxytocin (200 nmol/kg) increased the survival of experimental rats (FIG. 3A). The same effect was observed when fentanyl (1.3 mg/kg) is combined with xylazine (10 mg/kg) (FIG. 3B).

### SUMMARY OF EXAMPLES

These results indicate oxytocin is a promising treatment to mitigate opioid-induced respiratory depression in emergency and/or clinical settings. Building on the information disclosed herein, oxytocin's effectiveness could pave the way for improved interventions and better outcomes in combating the detrimental effects of the opioid crisis and the growing prevalence of xylazine in opioid use disorders requiring treatments instead of, or in combination with MOR antagonists.

What is claimed is:

1. A method of treating a subject, the method comprising:
administering to a subject experiencing xylazine-induced respiratory distress or alpha-2 agonist-mediated respiratory distress a therapeutically effective amount of a pharmaceutical formulation comprising oxytocin or an analog thereof and one or more pharmaceutically acceptable carriers.

2. The method of claim 1, wherein the subject is experiencing one or more symptoms associated with xylazine-induced respiratory distress or alpha-2 agonist-mediated respiratory distress.

3. The method of claim 2, wherein the one or more symptoms associated with the respiratory distress comprise decreased and/or reduced respiratory rate, tidal volume, minute ventilation, or any combination thereof.

4. The method of claim 1, wherein the subject is experiencing apnea.

5. The method of claim 1, wherein the analog is carbetocin, WAY-267,474, barusiban, or Antag III.

6. The method of claim 3, wherein, following the administering step, the respiratory rate, tidal volume, minute ventilation, or any combination thereof is increased and/or improved.

7. The method of claim 1, wherein administering comprises intranasal administration.

8. The method of claim 1, further comprising repeating the administering step.

9. The method of claim 1, wherein a therapeutically effective amount of the pharmaceutical formulation comprises at least about 1 IU to about 10 IU of oxytocin or the analog thereof.

10. The method of claim 1, further comprising administering to the subject one or more additional therapeutic agents.

11. A method of treating, reducing, and/or reversing respiratory distress, the method comprising:
administering to a subject experiencing respiratory distress a therapeutically effective amount of a pharmaceutical formulation comprising oxytocin or analogue thereof and one or more pharmaceutically acceptable carriers,
wherein the respiratory distress is xylazine-induced respiratory distress or alpha-2 agonist-mediated respiratory distress, and
wherein, following the administering step, one or more symptoms of the respiratory distress are treated, reduced, and/or reversed.

12. The method of claim 11, wherein the one or more symptoms associated with the respiratory distress comprise decreased and/or reduced respiratory rate, tidal volume, minute ventilation, or any combination thereof.

13. The method of claim 12, wherein, following the administering step, the respiratory rate, tidal volume, minute ventilation, or any combination thereof is increased and/or improved.

14. The method of claim 11, wherein the subject is experiencing apnea.

15. The method of claim 11, wherein administering comprises intranasal administration.

16. The method of claim 11, further comprising repeating the administering step.

17. The method of claim 11, wherein a therapeutically effective amount of the pharmaceutical formulation comprises at least about 1 IU to about 10 IU of oxytocin or the analog thereof.

18. The method of claim 11, wherein the analog is carbetocin, WAY-267,474, barusiban, or Antag III.

19. The method of claim 11, further comprising administering to the subject one or more additional therapeutic agents.
